(19) United States
(12) Patent Application Publication
(10) Pub. No.: US 2007/0032418 A1
(43) Pub. Date: Feb. 8, 2007

(54) Small-molecule inhibitors of angiogenin and rnases and in vivo and in vitro methods of using same

(75) Inventors: Robert Shapiro, Upton, MA (US); Jeremy L. Jenkins, Watertown, MA (US); Richard Y.T. Kao, Hong Kong (CN); Gary J. Latham, Austin, TX (US)

Correspondence Address: FULBRIGHT & JAWORSKI L.L.P., 600 CONGRESS AVE., SUITE 2400, AUSTIN, TX, 78701, US

(73) Assignee: Ambion, Inc, Austin, TX (US)

(21) Appl. No.: 10/546,855

(22) PCT Filed: Feb. 25, 2004
(86) PCT No.: PCT/US04/05663

§ 371 Date: Aug. 28, 2006

Related U.S. Application Data
(60) Provisional application No. 60/449,912, filed on Feb. 25, 2003.

(51) Int. Cl. A61K 38/22; A61K 31/655; A61K 31/555; A61K 31/409; C07D 487/22

(52) U.S. Cl. 514/12; 514/150; 514/185; 514/410; 514/563; 562/450; 534/816; 540/145

(57) ABSTRACT

Lead compounds were obtained in a high throughput screen (HTS) of angiogenin (ANG; a potent inducer of angiogenesis) enzyme activity, an RNase. One lead was shown to delay appearance of tumors in an animal tumor system, and to reduce the number of animals having tumors. Several lead compound analogs were found to be even more potent inhibitors of ANG activity compared to the original leads, and two were also found to have greater affinity for ANG than for pancreatic RNase. Other embodiments disclose a method comprising obtaining a ribonuclease inhibitor and a composition; and admixing the ribonuclease inhibitor and the composition to form an admixture, wherein a ribonuclease that may be present in the admixture is inhibited.

### RELATED APPLICATION

The present application claims the benefit of U.S. provisional application Ser. No. 60/449,912 filed Feb. 25, 2003, and which is incorporated herein by reference in its entirety.

### GOVERNMENT SUPPORT

This work was supported in part by Grant CA88738 from the National Institutes of Health, and the National Cancer Institute Developmental Therapeutics Program (R*A*N*D* project “Optimization of small-molecule lead compounds for inhibition of angiogenin”). The government has certain rights in the invention.

### TECHNICAL FIELD

The invention provides anti-tumor compositions which are lead compounds identified by high throughput screening and lead compounds having anti-tumor activity in vivo. This invention is also related to compositions and methods of inhibiting ribonucleases.

### BACKGROUND

Cancers of various types remain a leading cause of death worldwide. Current chemotherapeutic agents remain only a partial solution to control or cure a variety of cancers, and new anti-tumor agents are urgently needed. Inhibitors of the angiogenic factors, primarily proteins and protein fragments, that induce neo-vascularization to provide tumors with a blood supply have been studied.

Angiogenin (ANG), a single polypeptide chain of 123 amino acids, is a potent inducer of angiogenesis in vivo (Fett et al., 1985) that is secreted by tumor cells. The expression of ANG is elevated in many human cancers (Chopra et al., 1996; Chopra et al., 1997; Shimoyama et al., 1999; Shimoyama et al., 1999; Shimoyama et al., 1996; Eberle et al., 2000; Etoh et al., 2000). The available ANG antagonists, monoclonal antibodies, antisense oligonucleotides, and the ANG-binding protein actin, are effective in preventing the establishment and/or metastatic spread of multiple types of human tumors in athymic mice (Olson et al., 1994; Olson et al., 1995; Olson et al., 2002; Olson et al., 2001). These clinical and preclinical findings identify ANG as a potentially important target for new anticancer drugs. Although some of these agents may have utility as drugs in humans, small-molecule inhibitors would clearly be much more advantageous. Thus far, efforts to develop small antagonists have focused largely on an unusual aspect of ANG: its homology to bovine pancreatic RNase A. ANG is 33% identical to RNase A in sequence (Strydom et al., 1985) and its crystal structure reveals a similar fold, as well as some striking differences (Acharya et al., 1994; Leonidas et al., 1999). ANG exhibits a characteristic ribonucleolytic activity that differs markedly from that of RNase A and other related Rnases (Shapiro et al., 1986). This activity is necessary for the biological action of ANG (Shapiro and Vallee, 1989; Shapiro et al., 1989). Therefore, inhibitors of the enzymatic activity of ANG might be effective as anticancer drugs (Kao et al., 2002; Jenkins and Shapiro, 2003).

Previously, only nucleotides and related compounds were tested extensively for their capacity to inhibit the ribonucleolytic activity of ANG. The lowest Kᵢ values measured for these were only in the upper micromolar range at physiological pH (Russo et al., 1996; Russo et al., 2001; Kumar et al., 2003) and none of these compounds has been shown to have antitumor activity in vivo. Hence there is a compelling need to develop new low molecular weight ANG antagonists that bind sufficiently tightly to inhibit the tumor-promoting activity of ANG in vivo.

### SUMMARY OF THE INVENTION

A feature of an embodiment of the invention is a pharmaceutical composition comprising a compound selected from the group of: NCI-65828; benzopurpurin B; direct red 1; NCI-7815; NCI-45618; NCI-47740; NCI-58047; NCI-65553; NCI-65568; NCI-665534-P; NCI-65820; NCI-65841; NCI-65845; NCI-65847; NCI-79596; NCI-270718; NCI-724225; prBZBP; Chicago Sky Blue 6B; direct red 34; NCI-79741; xylidene ponceau 2R; amaranth; new coccine; acid red 37; acid violet 7; NCI-45608; NCI-73416; CB-102704; CB-128773; CB-140553; CB-180553; CB-180582; CB-0181431; CB-216112; CB-467929; CB-473872; JLJ-1; JLJ-2; and JLJ-3. The compound is present in an effective dose. In a related embodiment, the composition comprises a pharmaceutically acceptable buffer or salt.

The pharmaceutical composition in certain embodiments comprises an additional agent, for example, the additional agent is an anticancer agent. The additional agent is selected for example from at least one of the group of: paclitaxel; adriamycin; suramin; cisplatin; methotrexate; and 5-fluorouracil. Alternatively, the additional agent is a growth inhibitory factor or an anti-angiogenic protein, for example, the additional agent is endostatin or angiostatin. The antitumor composition in some embodiments comprises a pharmaceutically acceptable buffer. Further, the antitumor composition is provided in a unit dosage.

Another featured embodiment is a compound selected from the group of JLJ-1; JLJ-2; and JLJ-3. In a related embodiment, the compound is present in a composition comprising a pharmaceutically acceptable carrier, buffer or salt. The compound is present in an effective dose.

Another featured embodiment of the invention is a compound which is a derivative of NCI-65828, the compound having at least one modification selected from the group consisting an NCI-65828 having at least one of: a reduction of the azo to hydrazido; replacement of the azo by an amide; an attachment of a hydroxyl group or an electron-withdrawing group to position 6 of the naphthalene ring; replacement of a carbond atom in an aromatic ring with a nitrogen or an oxygen; and a replacement of the hydroxyl group on the biphenyl component with a sulfonate. For example, the derivative of NCI-65828 is a compound having at least one modification selected from the group consisting of: addition of a hydrogen-bonding group; and substitution of a hydroxyl group with an anionic group to the biphenyl component. The hydrogen-bonding group is selected from a hydroxyl, an amino, and an amide. The anion is selected from the group consisting of a carboxylate, a sulfate, a sulfonate, a phosphate, and a phosphonate.

Another featured embodiment of the invention is a derivative of CB-473872 having a modification which is an addition of at least one of a hydrogen-bonding group consisting of: a hydroxyl, an amino, an ethoxy, a methyldiamino, a hydroxymethyl, an ethyl-N-carboxyamido, formamido-N-ethyl, a carboxy, an 2-oxo-N-piperidinyl, and a p-benzoyl. Another featured embodiment of the invention is a derivative of CB-473872 having a modification which is an addition of at least one of a anionic group consisting of: carboxylate, a sulfate, a sulfonate, a phosphate, and a phosphonate.

Another featured embodiment of the invention is a compound which is a derivative of CB-473872 having Structure I as shown in FIG. 6, the derivative having at least one modification selected from the group consisting of: A compound which is a derivative of CB 473872, the derivative having Structure I and having at least one modification, the modification selected from the group consisting of: R₀ is —H, —NH₂, or —OH; R₁ is a —H, —CH₂OH, or —CH(NH₂)₂; R₂ is —H, —COOH, or —CH₂OH; R₃ is —H, —CH₂OH, or CONH₂; R₄ is —H, —COOH, or 2-oxo-N-piperidinyl; R₅ is —H or p-benzoyl; and replacement of a carbond atom in an aromatic ring with a nitrogen or an oxygen.

Another featured embodiment of the invention is a compound which is a derivative of CB-473872, the derivative having Structure II or structure III, and having at least one modification, the modification selected from the group consisting of: R0 is —H, —NH₂, or —OH; R3 is —H, —CH₂OH, or CONH2; R4 is —H, —COOH, or 2-oxo-N-piperidinyl; R5 is —H or p-benzoyl group; and replacement of a carbond atom in an aromatic ring with a nitrogen or an oxygen.

The invention also features a method for preventing or delaying tumor appearance and growth in a subject, comprising administering to the subject an inhibitor of angiogenin RNase activity. For example, the subject is a mammal, for example, the subject is a human. The tumor is selected from the group of cancers consisting; lung; breast; prostate; cervical; brain; head and neck; lymphoma; adenocarcinoma; sarcoma; and melanoma. Further, the tumor can be metastatic.

The invention also features a method for preventing or delaying progression of leukemia in a subject, comprising administering to the subject an inhibitor of angiogenin RNase activity. The leukemia is chronic; alternatively, the leukemia is acute.

The invention features, in a method of treating a tumor with an anti-angiogenic protein, an improvement comprising co-administering any of the anti-tumor compounds above, and an effective dosage of the anti-angiogenic protein, the effective dosage being lower than would be used in the absence of the compound. For example, the anti-angiogenic protein is endostatin or angiostatin.

The invention features a method of manufacture of a medicament for use in treating a subject having cancer, the method comprising manufacture of a medicament comprising an effective dose of a compound selected from the group of: NCI-65828; benzopurpurin B; direct red 1; NCI-7815; NCI-45618; NCI-47740; NCI-58047; NCI-65553; NCI-65568; NCI-665534-P; NCI-65820; NCI-65841; NCI-65845; NCI-65847; NCI-79596; NCI-270718; NCI-724225; prBZBP; Chicago Sky Blue 6B; direct red 34; NCI-79741; xylidene ponceau 2R; amaranth; new coccine; acid red 37; acid violet 7; NCI-45608; NCI-73416; CB-102704; CB-128773; CB-140553; CB-180553; CB-180582; CB-0181431; CB-216112; CB-467929; CB-473872; JLJ-1; JLJ-2; and JLJ-3.

The invention features a use of an effective dose of a compound selected from the group of: NCI-65828; benzopurpurin B; direct red 1; NCI-7815; NCI-45618; NCI-47740; NCI-58047; NCI-65553; NCI-65568; NCI-665534-P; NCI-65820; NCI-65841; NCI-65845; NCI-65847; NCI-79596; NCI-270718; NCI-724225; prBZBP; Chicago Sky Blue 6B; direct red 34; NCI-79741; xylidene ponceau 2R; amaranth; new coccine; acid red 37; acid violet 7; NCI-45608; NCI-73416; CB-102704; CB-128773; CB-140553; CB-180553; CB-180582; CB-0181431; CB-216112; CB-467929; CB-473872; JLJ-1; JLJ-2; and JLJ-3.

The invention features a kit for treating a cancer patient, comprising any of the above anti-tumor compounds above, in a container, and having instructions for use. Further in the kit, the compound in the container is present in a unit dosage.

Compounds of the invention—in addition to the therapeutic uses described above—are useful as ribonuclease inhibitors in a variety of molecular biology techniques. Therefore, in other aspects of the invention, there is provided a method comprising: (a) obtaining a ribonuclease inhibitor and a composition; and (b) admixing the ribonuclease inhibitor and the composition to form an admixture wherein a ribonuclease that may be present in the admixture is inhibited. The composition may include at least one ribonuclease. In other embodiments, the composition may include at least two, three, four, five, six, seven, or more ribonucleases. The composition may include a ribonucleic acid. In particular aspects, the ribonuclease inhibitor comprises a structure selected from the group consisting of NCI-65828, NCI 65845, benzopurpurin B, NCI-65841, NCI 79596, NCI-9617, NCI-16224, suramin, direct red 1, NCI-7815, NCI-45618, NCI-47740, prBZBP, NCI-65568, NCI-79741, NCI-65820, NCI-65553, NCI-58047, NCI-65847, xylidene ponceau 2R, eriochrome black T, amaranth, new coccine, acid red 37, acid violet 7, NCI-45608, NCI-75661, NCI-73416, NCI-724225, orange G, NCI 47755, sunset yellow, NCI-47735, NCI-37176, violamine R, NCI-65844, direct red 13, NCI-45601, NCI 75916, NCI-65546, NCI-65855, NCI-75963, NCI-45612, NCI-8674, NCI-75778, NCI-34933, NCI-1698, NCI-7814, NCI-45550, NCI-77521, cefsulodin, NCI-174066, NCI-12455, NCI-45541, NCI-79744, NCI-42067, NCI-45571, NCI-45538, NCI-45540, NCI-9360, NCI-12857, NCI-D726712, NCI-45542, NCI-7557, S321443, NCI-224131, NCI-45557, NCI-1741, NCI-1743, NCI-16163, NCI-16169, NCI-88947, NCI-227726, NCI-17061, NCI-37169, beryllon II, CB-0181431, CB-473872, JLJ-1, JLJ-2, JLJ-3, CB-467929, CB-534510, CB-540408, CB-180582, CB-180553, CB-186847, CB-477474, CB-152591, NCI-37136, NCI-202516, CB-039263, CB-181145, CB-181429, CB-205125, and CB-224197.

In particular aspects, the ribonucleases inhibitor may be a derivative of NCI-65828. The derivative of NCI-65828 may include at least one modification selected from the group consisting of: a reduction of the azo to hydrazido, replacement of the azo by an amide, an attachment of a hydroxyl group to position 6 of the naphthalene ring, an attachment of an electron-withdrawing group to position 6 of the naphthalene ring, replacement of a carbon atom in an aromatic ring with a nitrogen or an oxygen, and a replacement of the hydroxyl group on the biphenyl component with a sulfonate. In other embodiments, the derivative of NCI-65828 may include at least one modification selected from the group consisting of: an addition of a hydrogen-bonding group and substitution of a hydroxyl group with an anionic group to the biphenyl component.

In still another aspects, the ribonucleases inhibitor may be a derivative of CB-473872. The derivative of CB-473872 may include an addition of at least one of a hydrogen-bonding group selected from the consisting of: a hydroxyl, an amino, a methyldiamino, a hydroxyethyl, an ethyl-N-formamido, a carboxyamido, a carboxy, a 2-oxo-N-piperidinyl, and a p-benzoyl. In other embodiments, the derivative of CB-473872 comprises Structure I (see FIG. 6), wherein: R₀ is —H, —NH₂, or —OH; R₁ is a —H, —CH₂OH, or —CH(NH₂)₂; R₂ is —H, —COOH, or —CH₂OH; R₃ is —H, —CH₂OH, or CONH₂; R₄ is —H, —COOH, or 2-oxo-N-piperidinyl; and R₅ is —H or p-benzoyl.

The derivative of CB-473872 may include Structure II (see FIG. 7) or Structure III (see FIG. 8) wherein: R₀ is —H, —NH₂, or —OH; R₃ is —H, —CH₂OH, or CONH₂; R₄ is —H, —COOH, or 2-oxo-N-piperidinyl; R₅ is —H or p-benzoyl group. The derivative of CB-473872 may include a replacement of a carbon atom in an aromatic ring with a nitrogen or an oxygen.

The ribonuclease may be RNase A, RNase B, RNase C, RNase 1, RNase T1, micrococcal nuclease, S1 nuclease, DNase 1, a member of a mammalian ribonuclease A super family, a member of an RNase H family, RNase L, eosinophil RNase, messenger RNA ribonucleases, an E. coli endoribonuclease, an E. coli exoribonucleases, RNase Sa, RNase F1, RNase U2, RNase Ms, or RNase St. Other nucleases—such as DNases—may be inhibited by the methods and compounds disclosed in this invention.

In another embodiments, there is provided a compound comprising a structure selected from the group consisting of NCI-65828, NCI 65845, benzopurpurin B, NCI-65841, NCI 79596, NCI-9617, NCI-16224, suramin, direct red 1, NCI-7815, NCI-45618, NCI-47740, prBZBP, NCI-65568, NCI-79741, NCI-65820, NCI-65553, NCI-58047, NCI-65847, xylidene ponceau 2R, eriochrome black T, amaranth, new coccine, acid red 37, acid violet 7, NCI-45608, NCI-75661, NCI-73416, NCI-724225, orange G, NCI 47755, sunset yellow, NCI-47735, NCI-37176, violamine R, NCI-65844, direct red 13, NCI-45601, NCI 75916, NCI-65546, NCI-65855, NCI-75963, NCI-45612, NCI-8674, NCI-75778, NCI-34933, NCI-1698, NCI-7814, NCI-45550, NCI-77521, cefsulodin, NCI-174066, NCI-12455, NCI-45541, NCI-79744, NCI-42067, NCI-45571, NCI-45538, NCI-45540, NCI-9360, NCI-12857, NCI-D726712, NCI-45542, NCI-7557, S321443, NCI-224131, NCI-45557, NCI-1741, NCI-1743, NCI-227726, NCI-16163, NCI-16169, NCI-88947, NCI-17061, NCI-37169, beryllon II, CB-0181431, CB-473872, JLJ-1, JLJ-2, JLJ-3, CB-467929, CB-534510, CB-540408, CB-180582, CB-180553, CB-186847, CB-477474, CB-152591, NCI-37136, NCI-202516, CB-039263, CB-181145, CB-181429, CB-205125, and CB-224197.

In particular aspects of this invention, a nuclease inhibitor may include a compound comprising an aromatic structure. In other embodiments, the aromatic structure may be a polycyclic aromatic structure. A non-limiting example of nuclease inhibitors according to the invention include an aromatic structure of: 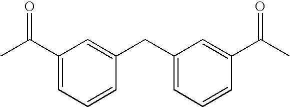
A further non-limiting example of nuclease inhibitors of the present invention include a polycyclic aromatic structure of:

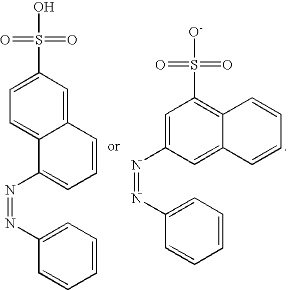

In some preferred embodiments, the nuclease inhibitors comprise the following structures:

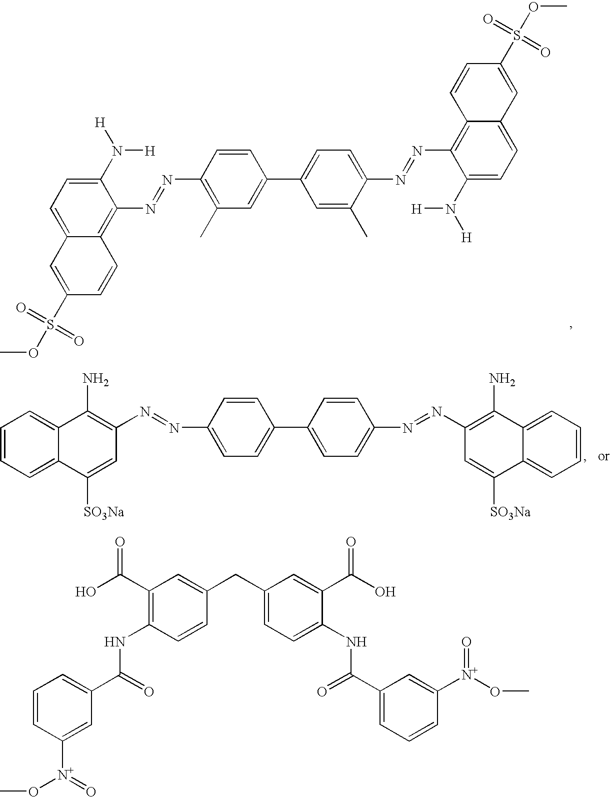

Modifications or derivatives of the above aromatic structures are also contemplated as being useful with the methods and compositions of the present invention. Non-limiting examples of modifications that can be made to these structures include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom.

The disclosed RNase inhibitors of the present invention can also be used in the practice of any number of molecular biology techniques known to those of skill in the art. Non-limiting examples include the techniques, kits, and reagent described in: (i) U.S. provisional application entitled “Nuclease Inhibitors for Use In Biological Applications” by Latham et al., filed on Feb. 25, 2004; and (ii) U.S. non-provisional application entitled “Improved Nuclease Inhibitor Cocktail” by Latham et al., filed on Feb. 25, 2004, which is a continuation-in-part application of co-pending U.S. application Ser. No. 10,675,860 filed Sep. 30, 2003, which is a continuation of application Ser. No. 09/669,301 filed Sep. 25, 2000, now U.S. Pat. No. 6,664,379, which claims the benefit of U.S. Provisional Application No. 60/155,874, filed Sep. 24, 1999. The entire text of each of the foregoing applications is specifically incorporated herein by reference without disclaimer.

### BRIEF DESCRIPTION OF THE DRAWINGS

FIG. 1 is a Kaplan-Meier survival plot showing delay or prevention of PC-3 (A and B) and HT-29 (C) tumor formation in athymic mice by NCI-65828 (Lead 1). Cells were injected s.c. on day 0, and treatment or control solutions (●, NCI-65828; ▪, vehicle; ▴, NCI-45557, a negative control compound) were administered locally s.c. 6×/week starting on the same day. Mice were examined 2×/week for palpable tumors. Treatment doses: 40 μg/day (A and C); 8 μg/day (B). n=8 for all groups except the vehicle controls in A and B, where n=12.

FIG. 2 is an atomic model of the complex of ANG (light) with NCI-65828 (Lead 1; dark) from modeling studies using the program AutoDock. Hydrogen bonds are denoted with broken lines.

FIG. 3 is an atomic model that shows the docked position of ChemBridge 181431 (Lead 2) and its relationship to the target protein angiogenin (ANG) catalytic residues His13, Lys40, and His114.

FIG. 4 shows the chemical structures of Lead 1 (NCI-65828) and Lead 2 (CB-181431), and those of four analogs of Lead 1 and one analog of Lead 2 that are also active ANG inhibitors. The Lead 2 analog CB-473872 is also referred to as Lead 2A.

FIG. 5 shows Lead 2 (CB-181431), Lead 2A (CB-473892), and also another Lead 2 analog, CB-467929, which has an m-nitrophenyl group rather than a fused ring (of Lead 2) or second ring structure (of Lead 2A), and also has a desirable Kᵢ of 24 μM.

FIG. 6 shows a set of Lead 2A derivatives designed by reference to a computational model of the three-dimensional structure of the complex of ANG and Lead 2A. In Structure I, a variety of molecular locations for derivatizing the structure are shown as R groups which can be independently derivatized (except for the box indicated “R₁+R₂”, in which case both positions are further derivatized) with any of the chemical groups shown in a box labeled as to the position to be derivatized, to improve interactions with indicated residues of ANG. In the various combinations, an alternative which is absence of derivatization indicates that the bond is —H.

FIG. 7 shows derivatives of Structure II, Structure II being the chemical structure of a derivative of Structure I having —(CH₂)₂NHCO substituted at R₁ and R₂.

FIG. 8 shows Structure III, Structure III being the chemical structure of a derivative of Structure I having —(CH₂)₂CO substituted at R₁ and R₂.

FIG. 9 shows that BpB offers significant protection from RNA degradation when challenged by a number of purified RNases, including E. coli RNase I. Lane 1: production source RNA control; lane 2: bovine RNase A control; lane 3: bovine RNase A+BpB; lane 4: EDN control; lane 5: EDN+BpB; lane 6: HPR control; lane 7: HPR+BpB; lane 8: RNase 1 control; lane 9: RNase I+BpB; lane 10: RNase T1 control; and lane 11: RNase T1+BpB.

FIG. 10 shows RNA challenge conditions: lane 1: production source RNA control; lane 2: RNase T1+Cpd #467929; lane 3: bovine RNase A control; lane 4: bovine RNase A+Cpd #467929; lane 5: HPR control; lane 6: HPR+Cpd #467929; lane 7: EDN control; lane 8: EDN+Cpd #467929; lane 9: RNase 1 control; lane 10: RNase 1+Cpd #467929; and lane 11: RNase T1 control.

### DETAILED DESCRIPTION OF EMBODIMENTS

A. Definitions

As used herein, the following words and phrases shall have the following definitions in the specification and in the claims.

An “anti-cancer” agent is any compound having a demonstrable activity, in vivo or in vitro or both, to promote death, necrosis, or apoptosis of a cancer cell, or diminishment in growth, appearance or symptom due to any cancerous or transformed condition affecting any cell, tissue, or tumor; or a compound which will prevent or reduce incidence of metastasis of a cancer; or a compound capable of causing remission of symptoms resulting from such cancerous condition. An anti-cancer agent can have broad specificity for numerous cancers, or can be specific for one or a few types of cancer. A newly discovered anti-cancer activity discovered for a known compound not previously associated with this compound is an anti-cancer agent as used herein.

An “anti-tumor agent” is defined as any composition having demonstrable activity, in vivo or in vitro, against any solid tumor, including lymphoma, and causing or promoting death, necrosis or apoptosis of a tumor cell, or delay in appearance or growth of a tumor. An anti-tumor agent that possesses anti-leukemic activity is an anti-cancer agent.

A “subject” shall mean a mammal, including without limitation, a human, a rodent such as a mouse or rat, a cow, a sheep, a goat, a horse, an ape, a monkey, a dog, a pig, or a cat.

A “modification” of a particular compound includes a closely related compound having, in one embodiment, one or more altered substituents on an otherwise identical or substantially similar atomic framework. Altered substituents include addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents.

In an additional embodiment, a “modification” can be an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom. A modification can be a “prodrug” derivative, which has significantly reduced pharmacological activity, and contains an additional moiety which is susceptible to removal in vivo yielding the parent molecule as the pharmacologically active species.

The term “analog” as defined herein is a compound that is similar or comparable, but not identical, to another compound having similar structural characteristics, but that is not identical in structure. As used herein, an analog is a chemical compound, for example, a peptide or a protein used as the target for drug discovery and thus included in HTS assays, that is structurally similar to another but differs slightly in composition (for example, a replacement of one atom by an atom of a different element, or a change in presence of a particular functional atom or group of atoms between the original compound and the analog).

The term “derivative” as used herein refers to a compound formed from the original structure either directly, by chemical reaction of the original structure, or by a “modification” which is a partial substitution of the original structure, or by design and de novo synthesis. Derivatives may be synthetic, or may be metabolic products of a cell or an in vitro enzymatic reaction. In general, a set of derivatives is synthesized from a lead compound obtained by HTS, in order to obtain compounds with improved properties of inhibition of the target used in the high throughput screen, the inhibition having been demonstrated by the lead compound. Additional derivatives can be synthesized in order to optimize other pharmacological properties of a lead compound, such as absorption, distribution, metabolism, and excretion or transformation into another compound.

The terms “inhibiting,” “reducing,” or “prevention,” or any variation of these terms, when used in the claims and/or the specification includes any measurable decrease or complete inhibition to achieve a desired result. “Inhibiting” does not require complete nuclease inactivation or even substantial nuclease inactivation. The term “substantial inhibition” connotes that there is no substantial degradation of DNA or RNA detected in a composition that may include DNA or RNA.

A “small molecule” refers to an organic molecule of less than 2,500 daltons, for example, less than 1,000.

“Docking” means the ability of a small molecule to fit to a feature of a protein surface such as a groove, cleft, or pocket formed by amino acids of the protein in a three-dimensional array by virtue of the small molecule and the protein having one or more of complementary sizes, shapes, and charges, and by the ability of the fit of surfaces to exclude water molecules. In one example, docking of a small molecule to a protein is assessed within a spherical area having a 12 Å radius. The better the fit obtained for docking of a small molecule docking to a target, the greater the predicted affinity or inhibitory effect is predicted for the small molecule on a reaction catalyzed by the protein, as assessed by Kₘ (or K_{D}), or Kᵢ, respectively. The structure of the target protein can be known through coordinates obtained, for example, preferably by crystallography, or by NMR, or other by procedures. The resolution of the locations of the atoms of the protein can be within distances of 2.5 Å, 2.0 Å, or 1.8 Å.

“High throughput screening” (HTS) refers to a plurality of assays that test a plurality of compounds, performed robotically, the results of which are generally measured electronically by changes in magnitude or wavelength maxima of absorption or emission of light for the purpose of finding a drug candidate (“hit”) among the compounds. In general, the assay measures an enzyme activity; and cleavage of a labeled substrate to a product causes a change in color, or wavelength of emission, or extent of emission, of such that multiple parallel samples can be read automatically. In general, multiwell plastic plates having at least 96 wells per plate, or 384 wells/plate, or 1536 wells/plate, are used in HTS. Because HTS is highly automated, it is generally performed on at least a plurality of compounds, for example, at least 1,000 compounds, for example, at least 2,000 compounds, at least 5,000 compounds, or at least 10,000 compounds. Libraries of compounds can be obtained from the NCI, from other agencies, and from commercial sources such as ChemBridge (San Diego, Calif.).

Fragments of plasminogen such as angiostatin and fragments of collagen XVIII such as endostatin are inhibitors of angiogenesis.

The terms “peptide”, “polypeptide” and “protein” as used herein shall have identical meaning.

B. Identification of Inhibitors of ANG Enzymatic Activity by High-Throughput Screening

The National Cancer Institute (NCI) Diversity Set (1,990 compounds each identified herein using the letters “NCI” with the number associated with each of the compounds, and which are described in other references as “NSC” compounds) and the ChemBridge DIVERSet™ (16,320 compounds; each identified herein using the letters “CB” with the number associated with each of the compounds), collectively referred to as “compounds”, were screened for inhibitory activity against ANG in an HTS assay measuring ANG-catalyzed cleavage of the fluorescent substrate 5′-FAM-mAmArCmAmA-C7-Dabcyl-3′ (Integrated DNA Technologies, Skokie, Ill.), where FAM is 6-carboxyfluorescein, rC is ribocytidine, mA is 2′-O-methyl-riboadenosine, and Dabcyl is 4(4-dimethylaminophenylazo)benzoic acid. This assay was adapted from a recently reported fluorescence-based assay performed in cuvettes (Kelemen et al., 1999). In the intact substrate, the fluorescence emission of FAM (λₑₓ=495 nm; λₑₘ=525 nm) is largely quenched by the Dabcyl group. Cleavage by ANG, which yields the products FAM-mAmArC cyclic 2′,3′-phosphate and mAmA-C7-Dabcyl, relieves the quenching and increases the fluorescence emission by ˜25-fold. The assay was conducted in 384-well plates, under conditions such that 7% of the substrate is cleaved in the absence of inhibitor. Fluorescence was measured with a Wallac 1420 Victor² multilabel counter (PerkinElmer Life Sciences, Wellesley, Mass.), and the median value on each plate was normalized to 1.0 FI (arbitrary unit) for analysis; this value is similar to that measured when no library compound is added. The background fluorescence in the absence of ANG was 0.2 FI. Test compounds that produced FT values <0.7 in the HTS were considered to be hits. For the NCI set, which was assayed at a final concentration of 25 μM (i.e., a 400-fold dilution from 10 mM stocks), this FI cutoff theoretically translates into an upper limit of 40 μM for Kᵢ. For the ChemBridge set, which was assayed at 20-70 μM (a 400-fold dilution of 5 mg/mL stocks), the Kᵢ values are predicted to be below 100 μM in all cases (median inhibitor concentrations and predicted Kᵢ limits were 29 μM and 46 μM, respectively).

HTS hits were tested by additional methods to determine which were true actives (defined as those compounds with Kᵢ values <100 μM); the reaction buffer (20 mM HEPES, pH 7.0, 0.1 M NaCl) was the same as that used for HTS. First, all of the hits were assayed for their capacity to inhibit ANG-catalyzed mRNA degradation; compounds that still appeared to be active were then subjected to detailed kinetic analysis with a highly-accurate HPLC-based assay. In the first assay, luciferase mRNA was incubated with ANG in the presence or absence of test compound, and then added to an in vitro translation system. The luciferase produced was quantified by adding luciferase substrate and measuring light output in a luminometer. The ANG concentration used, 60 nM, was sufficient to reduce luminescence by ˜70% in the absence of inhibitor. The compounds were tested at a concentration of 50 μM; those that restored luminescence to a level greater than that observed with an “inhibition control” having only 30 nM ANG (60% of ANG at the test concentration), in the absence of compound were defined as hits.

The HPLC assay used herein for final hit confirmation monitored cleavage of the ANG substrate 5′-(dA)₅-rC-(dA)₂-3′, where dA is 2′-deoxyriboadenosine, at a concentration well below Kₘ. Reaction mixtures with and without test compounds were chromatographed on a Mono Q anion-exchange column with an NaCl gradient in 10 mM Tris, pH 8. Remaining substrate and the product (dA)₅-rC cyclic 2′,3′-phosphate resolve fully from each other and from all test compounds in this system, and were quantified by peak area, monitoring absorbance at 254 nm. This information was used to calculate k_{cat}/Kₘ values, which were then fitted to the equation (k_{cat}/Kₘ)ᵢ=(k_{cat}/Kₘ)₀/(1+[I]Kᵢ), where (k_{cat}/Kₘ)ᵢ and (k_{cat}/Kₘ)₀ are defined as the values in the presence and absence of inhibitor, respectively, and [I] is defined as the inhibitor concentration. At least four concentrations (in a range of about 10 μM-above 100 μM) were used in the final hit confirmation in order to distinguish all true hits.

Fifteen of the 18,310 compounds screened were confirmed as inhibitors of the enzymatic activity of ANG with Kᵢ values less than 100 μM. Two of these were considered particularly suitable as leads for further investigation, based on their affinity for ANG and their chemical properties. These compounds are: (i) NCI-65828 (Lead 1; 8-amino-5-(4′-hydroxybiphenyl-4-ylazo)naphthalene-2-sulfonate; Kᵢ=81 μM) and (ii) CB-181431 (Lead 2; 4,4′-dicarboxy-3,3′-bis(naphthylamido)-diphenylmethanone; Kᵢ=41 μM).

C. Ribonucleases (RNases)

Non-limiting examples of ribonucleases that can be inhibited by using the present invention include: RNase A, RNase B, RNase C, RNase 1, RNase T1, micrococcal nuclease, S1 nuclease, or DNase 1. Additional eukaryotic ribonucleases that can be inactivated include, but are not limited to, a member of a mammalian ribonuclease A super family (i.e., ribonucleases 1-8), a member of an RNase H family, RNase L, eosinophil RNase, messenger RNA ribonucleases (5′-3′ Exoribonucleases, 3′-5′ Exoribonucleases), decapping enzymes and deadenylases. Other non-limiting ribonucleases that can be inhibited and/or inactivated by the methods and compositions of the present invention include E. coli endoribonucleases (RNase P, RNase III, RNase E, RNase I, RNase HI, RNase HII, RNase M, RNase R, RNase IV, F; RNase P2, O, PIV, PC, RNase N), E. coli exoribonucleases Abase II, PNPase, RNase D, RNase BN, RNase T, RNase PH, OligoRNase, RNase R), RNase Sa, RNase F1, RNase U2, RNase Ms, and RNase St. Both endonucleases and exonucleases can be inhibited by the compositions and methods of the present invention. One of skill in the art can readily employ the methods and compositions of the present invention to inhibit and/or inactivate other RNases known in the art beyond those specifically named.

In certain aspects of this invention, the disclosed methods and compositions can be used to target and inhibit the activity of a selected RNase without affecting the activity of a non-selected RNase. In other embodiments, the methods and compositions can be used to target and inhibit the activity of multiple RNases. These embodiments may be useful in the context of molecular biological embodiments and therapeutic embodiments.

D. Methods and Uses

A pharmaceutically acceptable carrier includes any and all solvents, dispersion media, coatings, antimicrobials such as antibacterial and antifungal agents, isotonic and absorption delaying agents and the like that are physiologically compatible. Preferably, the carrier is suitable for oral, intravenous, intramuscular, intraperitoneal, transdermal, or subcutaneous administration, and the active compound can be coated in a material to protect it from inactivation by the action of acids or other adverse natural conditions.

A composition of the present invention can be administered by a variety of methods known in the art as will be appreciated by the skilled artisan. The active compound can be prepared with carriers that will protect it against rapid release, such as a controlled release formulation, including implants, transdermal patches, micro-encapsulated delivery systems.

Many methods for the preparation of such formulations are patented and are generally known to those skilled in the art. See, e.g., Sustained and Controlled Release Drug Delivery Systems, J. R. Robinson, Ed. Marcel Dekker, Inc., NY (1978).

Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration. Dosage regimens can be adjusted to provide the optimum desired response (e.g., a therapeutic response). For example, a single bolus can be administered, several divided doses can be administered over time, or the dose can be proportionally reduced or increased as indicated by the exigencies of the disease situation.

In general, a preferred embodiment of the invention is to administer a suitable daily dose of a therapeutic composition that will be the lowest effective dose to produce a therapeutic effect, for example, mitigation of symptoms such as inhibiting growth of a tumor or causing regression in size of the tumor. The therapeutic compounds of the invention are preferably administered at a dose per subject per day of at least 2 mg, at least 5 mg, at least 10 mg or at least 20 mg as appropriate minimal starting dosages. In general, the compound of the effective dose of the composition of the invention can be administered in the range of 50 to 400 micrograms of the compound per kilogram of the subject per day.

A physician or veterinarian having ordinary skill in the art can readily determine and prescribe the effective dose of the pharmaceutical composition required. For example, the physician or veterinarian could start doses of the compound of the invention employed in the pharmaceutical composition at a level lower than that required in order to achieve the desired therapeutic effect, and increase the dosage with time until the desired effect is achieved.

In another preferred embodiment, the pharmaceutical composition includes also an additional therapeutic agent. Thus in a method of the invention, the pharmaceutical composition can be administered as part of a combination therapy, i.e. in combination with an additional agent or agents. Examples of materials that can be used as combination therapeutics with the compounds for treatment of tumors and cancer conditions as additional therapeutic agents include: an antibody or an antibody fragment that can bind specifically to a protein on a cancer cell such as HER-2 or CEA; a bispecific antibody capable of binding to a cancer call and effecting lysis by a macrophage; a chemotherapeutic agent such as 5-fluorouracil, methotrexate, paclitaxel, suramin, cisplatin, or adriamycin; a growth inhibitory peptide; an inhibitor of neovascularization, i.e., an anti-angiogenesis agent, for example, a protein such as endostatin or angiostatin; or an anti-microbial agent such as an antibiotic, an antifungal agent, or an antiviral agent.

An improvement in the symptoms as a result of such administration is noted by a reduction in tumor size or disappearance of the tumor; or reduction in appearance or growth of tumors. A therapeutically effective dosage preferably reduces tumor growth or metastasis by at least about 20%, more preferably by at least about 40%, even more preferably by at least about 60%, and even still more preferably by at least about 80%, relative to untreated subjects.

Embodiments of the invention having now been fully described, are further illustrated by the following examples, which are included for illustrative purposes and are not to be construed as limiting. Unless otherwise defined, all technical and scientific terms used herein have the same meaning as commonly understood by a person of ordinary skill in the art to which this invention belongs. Although methods and materials similar or equivalent to those described herein can be used in the practice or testing of the present invention, suitable methods and materials are described below. All publications, patent applications, patent, and other references mentioned herein are incorporated by reference in their entirety. In the case of conflict, the present specification, including definitions, will control. In addition, the materials, methods, and examples are illustrative only and not intended to be limiting. Some of the data have been published in a paper, “Identification of Small-Molecule Inhibitors of Human Angiogenin and Characterization of Their Binding Interactions Guided by Computational Docking”, by Jeremy L. Jenkins and Robert Shapiro, in the journal Biochemistry (volume 42, pages 6674-6687, 2003; publication dates May 13, 2003 (online) and Jun. 10, 2003 (print), the contents of which are expressly incorporated herein by reference in their entirety.

### EXAMPLES

### Example 1

### Criteria for the Analysis of Nuclease Inactivation

The inventors routinely perform assays on RNA and DNA designed to assess RNase and DNase activity in a sample. Many assays may be used for the detection of nuclease activity, including isotopic and non-isotopic assays. The assays generate similar data with regard to the sensitivity of detection.

In such assays, the inactivation process can be performed, for example, on a mixture of three different ribonucleases: RNase A, RNase 1, and RNase T1. Each ribonuclease may be purified from a different species: human, E. coli and fungal, respectively. The three RNases are very different from each other in their origin, substrate specificity, and protein sequence. In this way, the inactivation process can test three completely different but well characterized ribonucleases.

By employing assays, one of skill will be able to determine additional anti-nuclease antibodies, small molecules, proteinaceous compounds and organic compounds that function in the invention. In order to do so, one need only obtain a sample compound or small molecule that is expected to have nuclease inactivating activity and then perform the types of assays performed herein to determine the utility of the sample compound or small molecule in the methods and compositions of the invention.

Isotopic Assay: The isotopic RNase assay uses a radioactive RNA synthesized by in vitro transcription of the RNA substrate. The radioactive RNA is synthesized using a T7 MAXscript™ transcription kit (Ambion, Inc.). The in vitro transcription reaction mixture may contain, for example, 1.0 μg of linearized DNA template, 2 μl of 10× transcription buffer, 0.02 μl of UTP[α-³²P] (800 Ci/mmol), 2 μl of each 10 mM ribonucleotide, and 2 μl of the T7 RNA polymerase mix, with a final volume of 20 μL. The reaction can be incubated at 37° C. for a period of time ranging from 5 min to 24 hours. In particular embodiments, the reaction is incubated for 30 min. The transcript is purified by phenol:chloroform extraction and used directly for RNase inactivation assay (2.2×10⁵ counts per minute (approximate specific activity of the probe)/2.3 ng RNA).

Two μl of the RNA probe are incubated with the test sample in a final volume of 10 μl for about 16 hours at 37° C. After incubation, the RNA is fractionated in a denaturing 6 M urea 5% acrylamide gel. The gel is then exposed to x-ray film. Untreated RNA is also fractionated as control with the test samples for comparative purposes. Test samples containing no detectable RNase activity produce the same single band as the untreated control RNA. RNase activity is indicated by the intensity of the RNA decreasing and by the appearance of smearing below the intact RNA.

Non-Isotopic Assay: The non-isotopic assay can use total RNA isolated from mouse as the substrate for the RNase mixture. The assays are typically performed in a final volume of 10 μl. A quantity of 4 μg of total RNA isolated from mouse liver or brain is dissolved in an aqueous solution, usually water or 1 mM sodium citrate (pH 6.6). The treated ribonuclease sample is added to the total RNA and then incubated at 37° C. for a period of time ranging from 5 min to 24 hours, depending on the sensitivity desired for the assay. In particular embodiments, the reaction is incubated for 1 to 16 hours. After incubation, the RNA is fractionated in a formaldehyde 1% agarose gel. The RNA can be detected by staining with ethidium bromide and then illuminating the gel with ultraviolet light. The RNA fluoresces in the gel. Untreated total RNA can also be fractionated as a control with the test samples for comparative purposes. Test samples containing inactivated RNase produced the same ethidium bromide staining pattern as the untreated RNA control. Intact total RNA has two major bands produced by the 28S and 18S ribosomal RNA. If the intensities of the ribosomal RNAs become diminished compared to the control RNA, then the RNases were not inactivated by the inactivation treatment.

One of skill in the art can employ the same type of methods disclosed above, appropriately adapted, to assay for inactivation of DNase. For example, assays for analysis of the DNA degrading activities of nuclease S1, Micrococcal nuclease, and DNase 1 are contemplated.

### Example 2

### Initial Tests of Lead Compounds in an Animal Model System

Compound NCI-65828 was identified by HTS as an ANG inhibitor. This compound, or a control compound known to have low ANG inhibitory activity (NCI-45557 100 μL) was injected subcutaneously 10 min after an injection of human prostate tumor cells (PC-3), into in the same area of the back of athymic mice. Each compound was administered 6 times per week, in doses of 40 μg, 8 μg, and 1.6 μg (about 2, 0.4 and 0.08 mg/kg/day). The compound was dissolved in DMSO, and control mice received a corresponding volume of DMSO (4%, 0.8% and 0%). Another control group received saline. Mice receiving compound NCI-65828 in this preliminary test developed tumors at a slower rate compared to control groups.

Another in vivo mouse experiment was performed using 8 or 12 mice per group. The experimental group was administered PC-3 cells and NCI-65828, as intraperitoneal injections (40 μg and 8 μg). A control group of mice was administered PC-3 cells and a related compound that was known to be ineffective at inhibiting ANG enzymatic activity. Mice were evaluated for number of animals that remained tumor free as a function of time, at intervals of 3 days from days 14-42 following the initial injection.

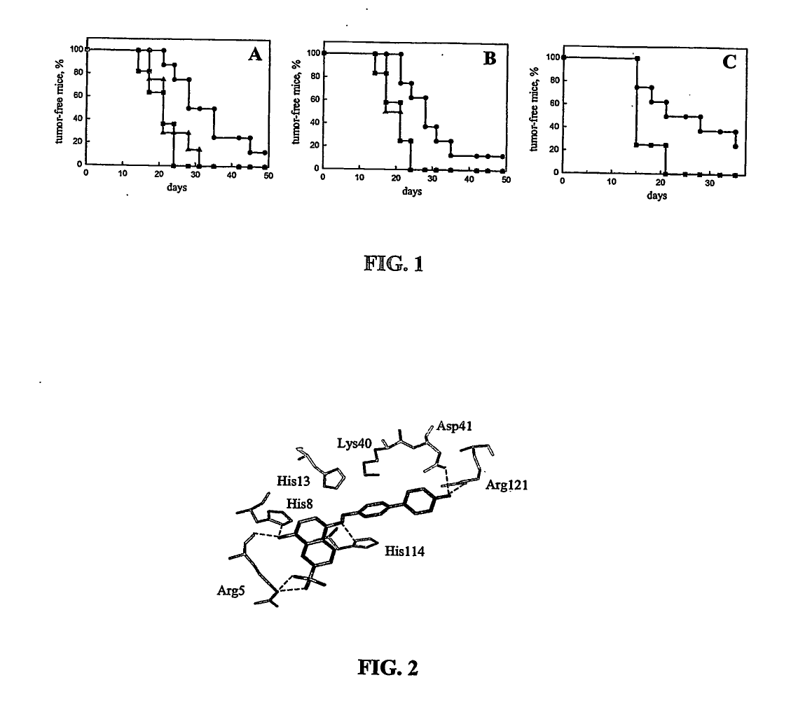

Results are displayed in FIGS. 1A and 1B, which show that administration of NCI-65828 (Lead 1), at each of the two doses, delayed the appearance of tumors in mice, and reduced the number of mice having tumors compared to controls not administered NCI-65828, during the period from about day 21 through the last time point of monitoring the tumors, day 42.

Further, local treatment with modest doses of NCI-65828 significantly delayed the formation of tumors that developed following administration of HT-29 human colonic adenocarcinoma cells (FIG. 1C). These data show effectiveness of an ANG inhibitor for treating subjects carrying each of two different cancer cell lines of human origin.

Additional data on in vivo effects are found in Kao et al., (2002), the contents of which are incorporated by reference herein in their entirety.

### Example 3

### Tests of Purified NCI-65828 in an Animal Model System

The test compounds were as follows. NCI-65828 in Table 1 refers to the 65828 preparation provided by NCI, which was used in Example 2 above. The Kᵢ for inhibition by NCI-65828 of the ribonucleolytic activity of angiogenin assayed as described herein is 81 μM (in 20 mM Hepes, 100 mM NaCl, pH 7.0, 37° C.). This material showed significant efficacy in the Example 2 tumor experiment, however, as the preparation was known to be crude rather than highly purified, another preparation which is highly purified was sought, to determine whether the activity would be retained by the purified preparation of NCI-65828.

TABLE 1
Effects of Angiogenin Inhibitors on Growth of Human Tumor Cells in Athymic
Mice-Percentage of Tumor-Bearing Mice as a Function of Time

| | | | | | | | # mice | |
|---|---|---|---|---|---|---|---|---|
| | day 14 | day 17 | day 21 | day 24 | day 32 | day 35 | per group | P value |
| PBS control | 12.5 | 62 | 62 | 88 | 100 | 100 | 8 | |
| 4% DMSO control | 25 | 88 | 100 | 100 | 100 | 100 | 8 | |
| 0.8% DMSO control | 50 | 88 | 100 | 100 | 100 | 100 | 8 | |
| Antisense control | 0 | 0 | 0 | 0 | 0 | 0 | 4 | 0.0019 |
| NCI-65828, 40 μg | 0 | 25 | 50 | 100 | 100 | 100 | 8 | 0.0052 |
| NCI-65828, 8 μg | 0 | 38 | 50 | 100 | 100 | 100 | 8 | 0.0056 |
| Tyger-65828, 40 μg | 8 | 33 | 33 | 83 | 92 | 100 | 12 | 0.0037 |
| Tyger-65828, 20 μg | 0 | 25 | 33 | 92 | 100 | 100 | 12 | 0.0008 |
| Tyger-65828, 8 μg | 0 | 45 | 55 | 82 | 91 | 100 | 11 | 0.0037 |
| Tyger-65828, 4 μg | 8 | 42 | 67 | 100 | 100 | 100 | 12 | 0.0098 |

The compound identified in Table 1 as “Tyger-65828” was synthesized by Tyger Scientific (Princeton, N.J.; custom synthesis); identity and purity were established by NMR, HPLC, TLC, MS and elemental analysis. The Kᵢ of Tyger-65828 for inhibition of the ribonucleolytic activity of angiogenin is indistinguishable from that of the NCI compound.

Concentrated stocks of inhibitors were prepared in dimethylsulfoxide (DMSO), and the inhibitors were diluted into phosphate-buffered saline (PBS); the final DMSO concentration for the 40 μg and 20 μg doses was 4% in PBS, and that for the 8 μg and 4 μg doses was 0.8% in PBS. Statistical significance (P values) was determined from Mantel-Cox tests performed on Kaplan-Meier survivor functions. Results for inhibitor-treated groups were compared to results obtained with the appropriate DMSO control groups. A positive control group of mice, administered antisense DNA specific for the ANG gene in PBS (no DMSO), was also included. ANG antisense DNA was shown previously to be extremely effective in this tumor model.

Tumor cells from a human prostate cancer (PC-3) were injected subcutaneously (s.c.) in the shoulder of 6-7 week old athymic (nude) mice on day 0. Treatment with the indicated amount of inhibitor (in 100 μL) was given subcutaneously in the same area as the cells, 6 times per week, with the first injection 5-10 minutes prior to injection of the cells. The total dosage was 0.4 mg to 2 mg per kg per day.

Tumors were measured on the days indicated in Table 1, as is data showing the presence or absence of tumors at each day. Administration of each of the original preparation of NCI-65828, and the highly pure Tyger-65828 preparation, delayed considerably the time of appearance of tumors, and also reduced the total number of animals having tumors. These data show that the highly purified material is at least as active as the original preparation, hence activity of this compound is not attributable to an unrelated contaminant material in the original preparation, nor to a synergistic interaction of NCI-65828 and another composition.

The positive control (anti-ANG antisense DNA) prevented appearance of tumors. The negative control mice (receiving PBS; DMSO; or inactive compound NCI-4557) developed tumors at an earlier time point and at greater frequency per mouse than those receiving either the original preparation of NCI-65828, or the highly pure Tyger-65828 preparation. These data also indicate that the mouse tumor system herein was effective in distinguishing anti-tumor activity from these controls.

Further, the data here confirm those in Example 2, and indicate that lead compound NCI-65828 is a successful anti-tumor agent in an animal model of cancer.

### Example 4

### Analogs of Lead Compounds

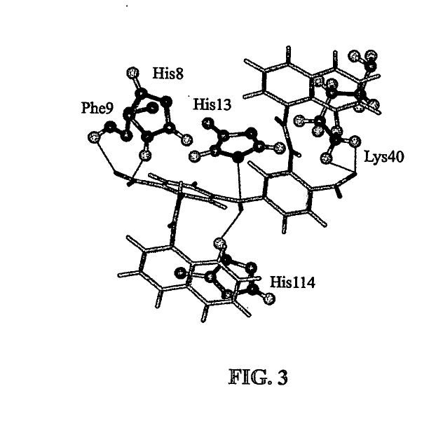

Models of complexes of ANG with each of the leads NCI-65828 (Lead 1) and CB-181431 (Lead 2) were generated with the program AutoDock (Morris et al., 1998) using the crystal structure of ANG in complex with inorganic phosphate (Leonidas et al., 2001). The models (described in detail in Jenkins and Shapiro, Biochemistry 42, 6674-6687, 2003, the contents of which are incorporated herein by reference in their entirety) indicated that the azonaphthalenesulfonic acid portion of NCI-65828 forms key interactions with the ANG active site (FIG. 2), and that, for Lead 2, predicted that key interactions with ANG involve the carboxylate and amide of one of the half-molecules (FIG. 3). The full NCI and ChemBridge libraries were examined to identify analogs that retained the functional groups predicted to be most important for recognition, and also for control compounds that lack these groups.

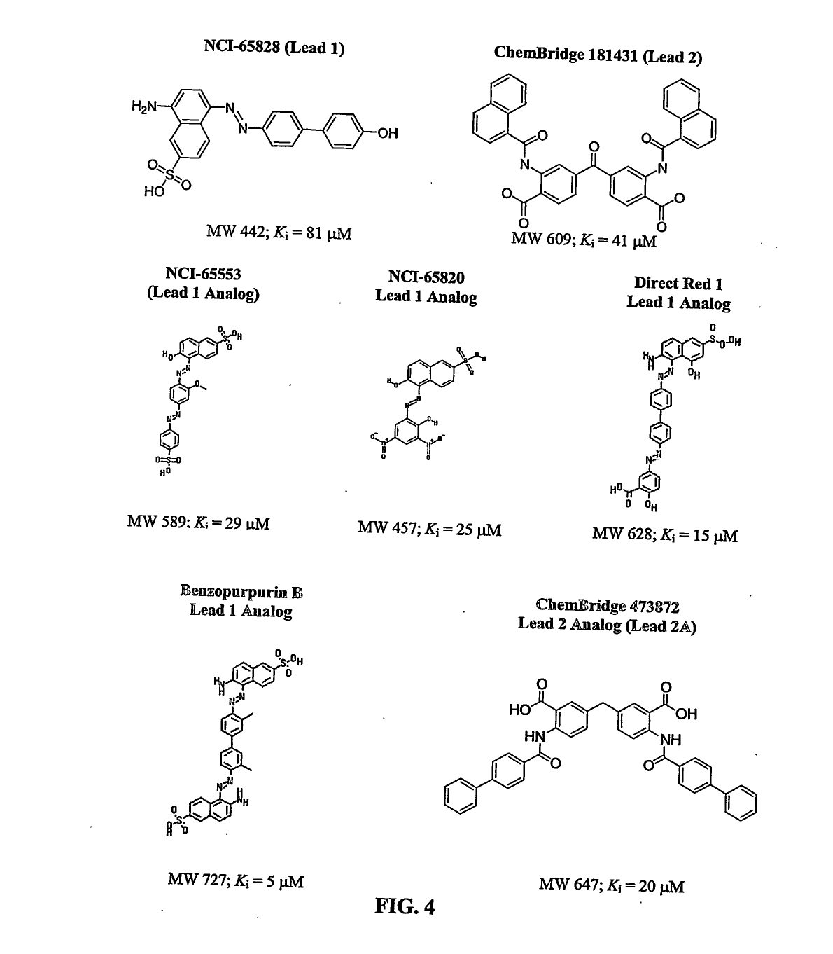

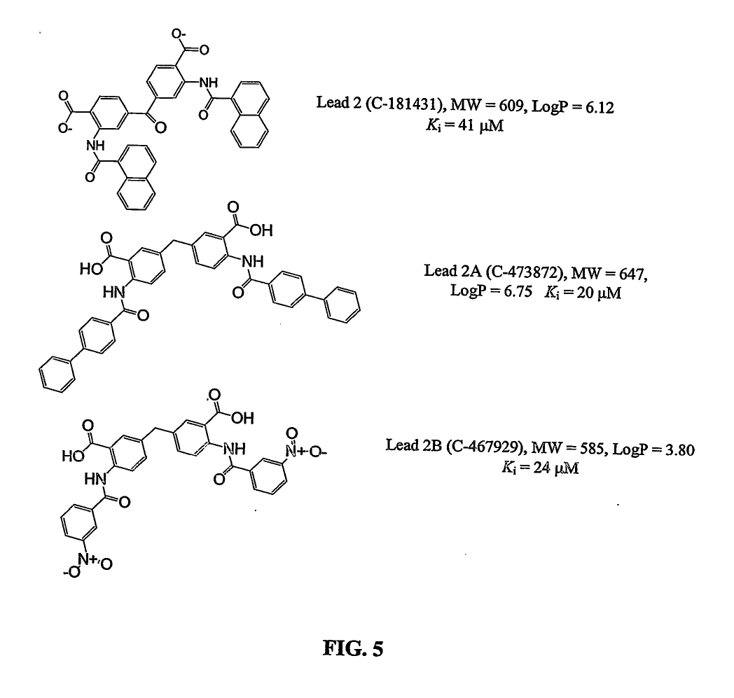

The analogs tested (sixty for Lead 1, NCI-65828, and nine for Lead 2, CB-181431) are listed in Tables 2 and 3 together with their K; values as determined from inhibition of ANG-catalyzed cleavage on an RNA oligonucleotide in 20 mM Hepes, 100 mM NaCl, and at 37° C. Chemical structures of selected compounds are shown in these Tables and in FIGS. 4 and 5. Three additional Lead 2 analogs and one additional Lead 1 analog, not available from NCI or commercial sources, were synthesized and tested as well; these compounds (JLJ-1, JLJ-2, JLJ-3, and prBZBP) are also included in Tables 2 and 3.

TABLE 2
Lead 1 Analog List

| Li- | | | | MW | |
|---|---|---|---|---|---|
| brary | Structure | # | K_{I} | pH7 | LogP |
| NCI | 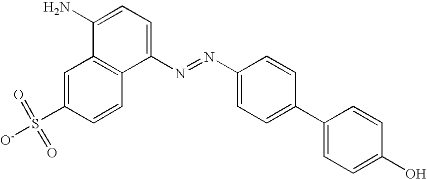 | 65828 | 81 | 418 | 3.47 |
| NCI | 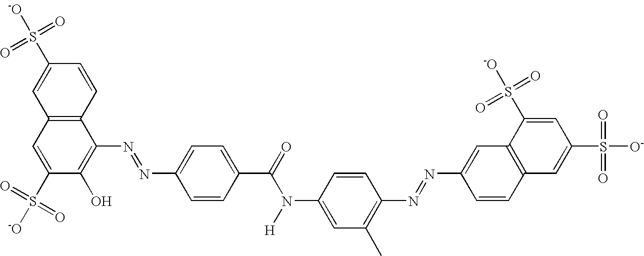 | 65845 | 3 | 852 | 2.70 |
| Rare Ald- rich & NCI | 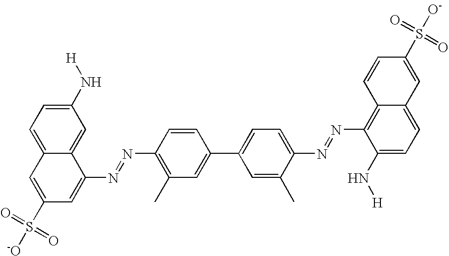 | Benzopur- purin B/ 242027 | 5 | 679 | 5.25 |
| NCI | 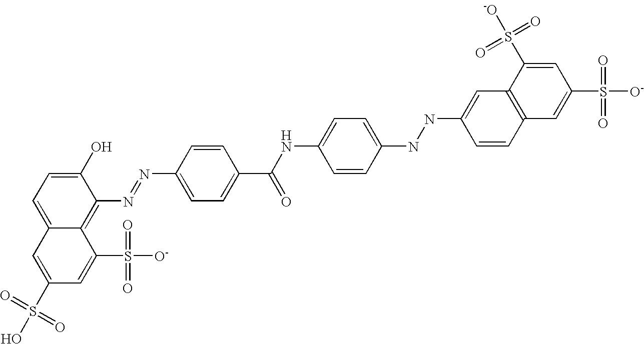 | 65841 | 5 | 839 | 2.28 |
| NCI | 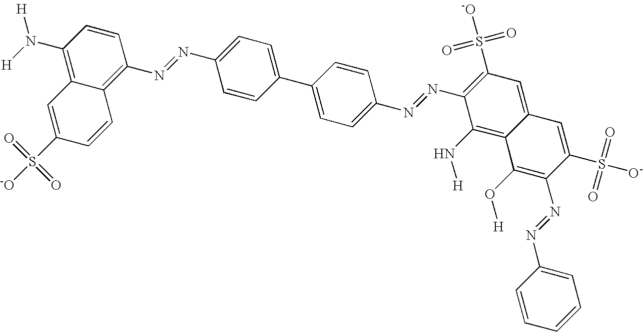 | 79596 | 5 | 850 | 4.28 |
| NCI | 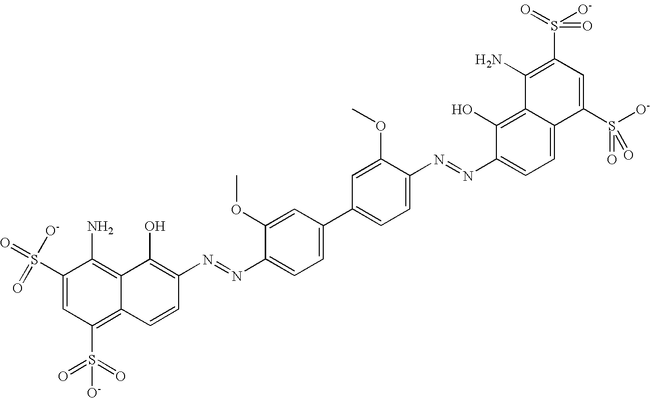 | 9617/ Chicago Sky Blue 6B | 5 | 901 | 1.26 |
| NCI Re- serve | 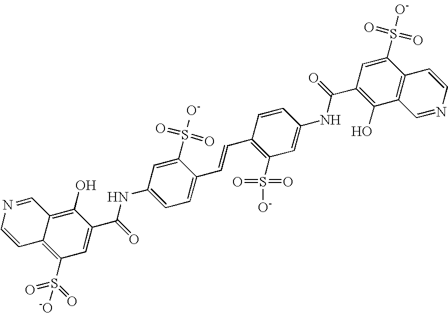 | 665534-P | 5 | 869 | −0.61 |
| NCI | 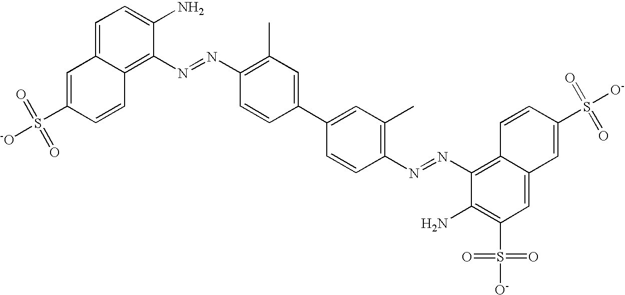 | 16224/ direct red 34 | 5.5 | 761 | 3.93 |
| Sig- ma- Ald- rich | 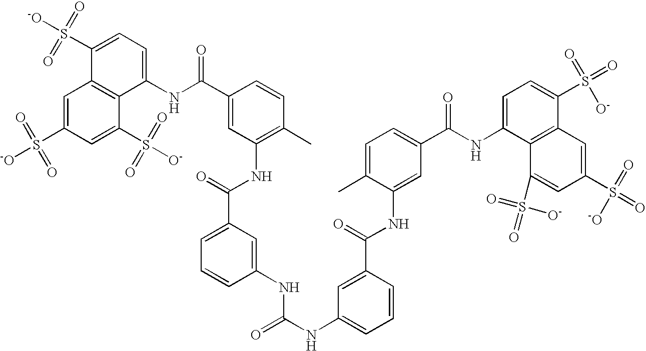 | suramin | 10 | 1291 | −6.26 |
| Rare Ald- rich & NCI | 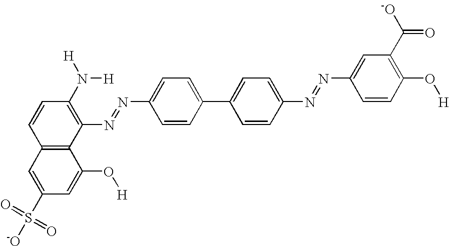 | direct red 1/ N-73358 | 14 | 582 | 4.02 |
| NCI | 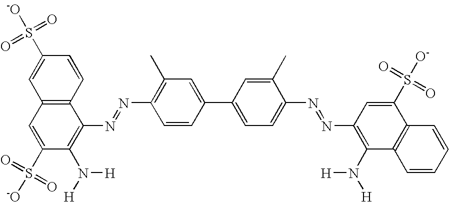 | 7815 | 14 | 758 | 3.93 |
| NCI | 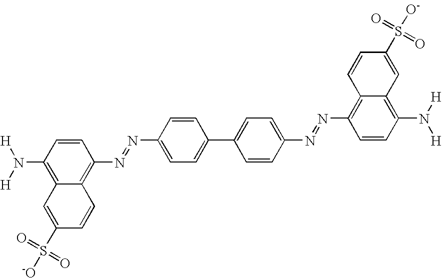 | 45618 | 15 | 651 | 4.42 |
| NCI | 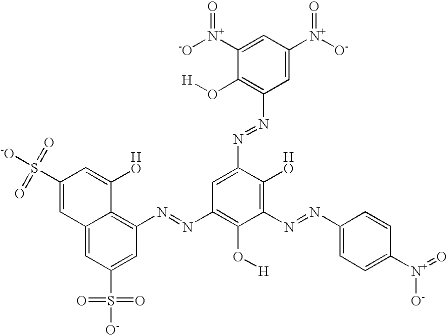 | 47740 | 15 | 798 | 3.39 |
| New | 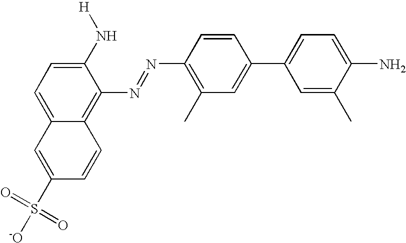 | prBZBP | 20 | 445 | 3.80 |
| NCI | 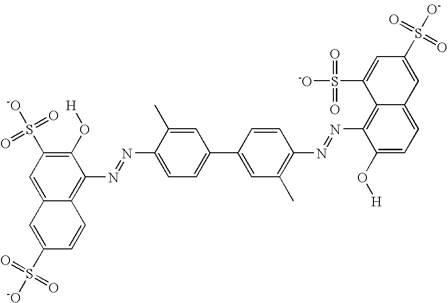 | 65568 | 23 | 839 | 3.45 |
| NCI | 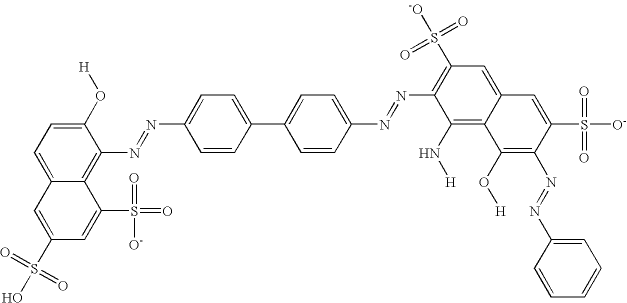 | 79741 | 23 | 931 | 3.39 |
| NCI | 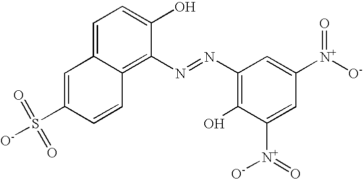 | 65820 | 25 | 433 | 2.38 |
| NCI | 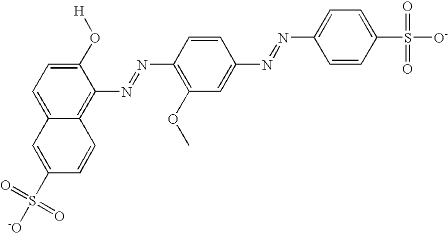 | 65553 | 29 | 541 | 3.23 |
| NCI | 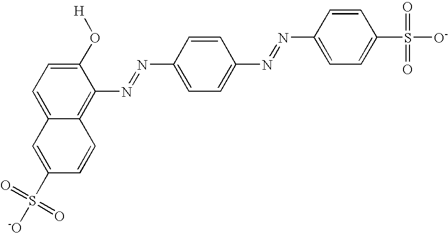 | 58047 | 36 | 511 | 3.10 |
| NCI | 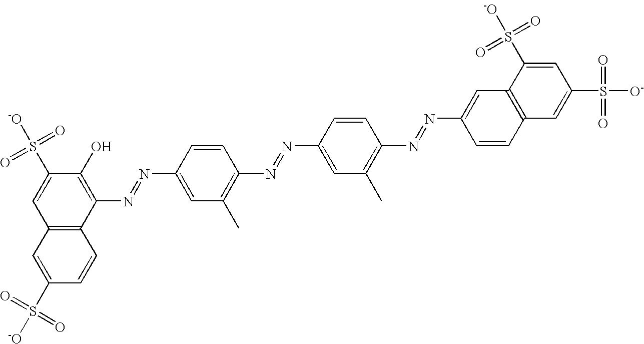 | 65847 | 38 | 851 | 3.98 |
| Sig- ma- Ald- rich | 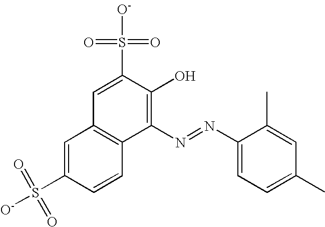 | xylidene ponceau 2R | 49 | 434 | 2.36 |
| Sig- ma- Ald- rich | 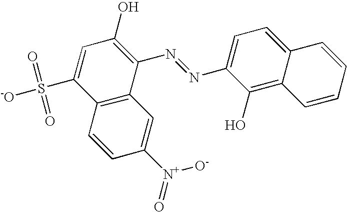 | eriochrome black T | 50 | 438 | 3.54 |
| Sig- ma- Ald- rich | 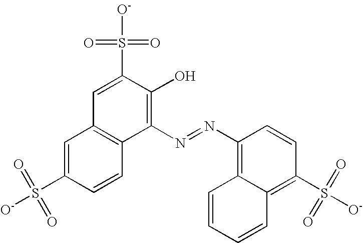 | Amaranth | 60 | 536 | 1.32 |
| Sig- ma- Ald- rich | 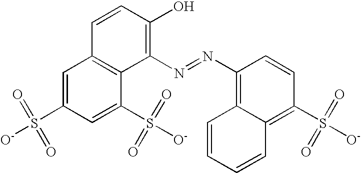 | new coccine | 69 | 536 | 1.32 |
| Sig- ma- Ald- rich | 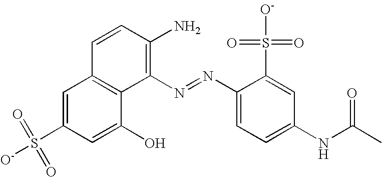 | acid red 37 | 70 | 478 | 0.13 |
| Sig- ma- Ald- rich | 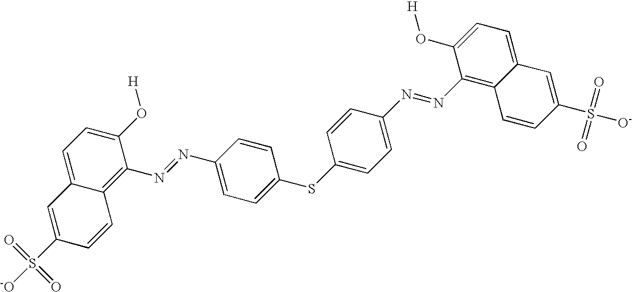 | acid violet 7 | 71 | 441 | 1.68 |
| NCI | 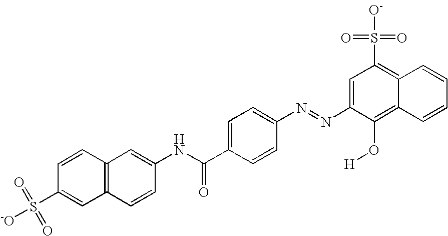 | 45608 | 75 | 576 | 3.35 |
| NCI | 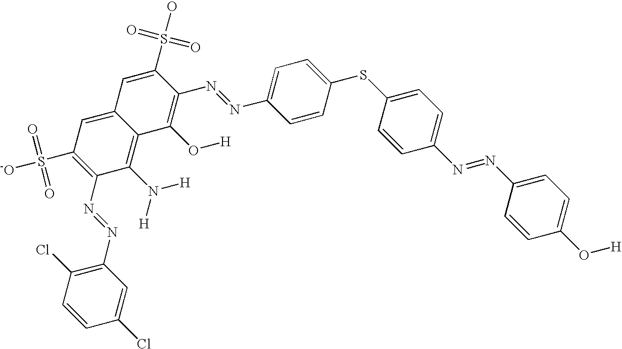 | 75661 | 76 | 823 | 7.03 |
| NCI | 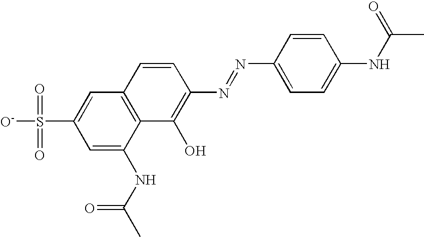 | 73416 | 77 | 685 | 6.15 |
| NCI | 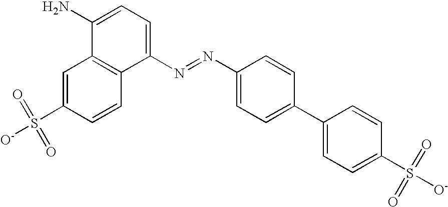 | 724225 | 81 | | |
| Sig- ma- Ald- rich | 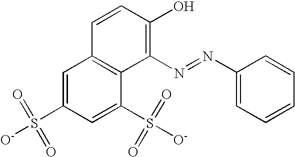 | Orange G | 83 | 406 | 1.53 |
| NCI | 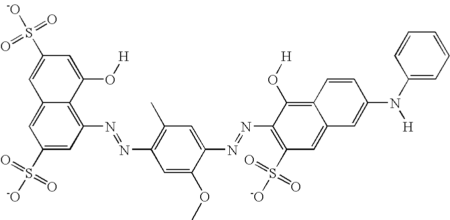 | 47755 | 84 | 791 | 4.65 |
| Sig- ma- Ald- rich | 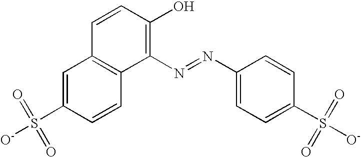 | sunset yellow | 85 | 406 | 1.53 |
| NCI | 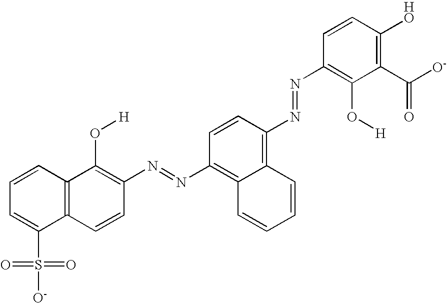 | 47735 | 85 | 557 | 4.13 |
| NCI | 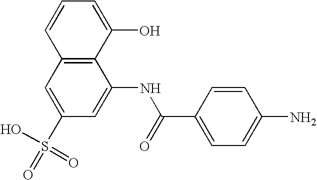 | 37176 | 104 | 358 | 1.80 |
| Rare Ald- rich | 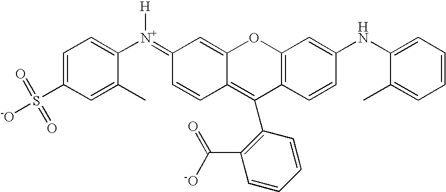 | violamine R | 107 | 590 | 6.19 |
| NCI | 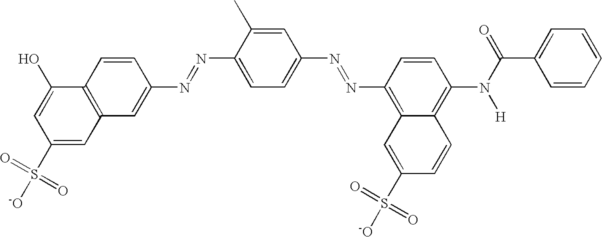 | 65844 | 108 | 694 | 5.34 |
| Rare Ald- rich | 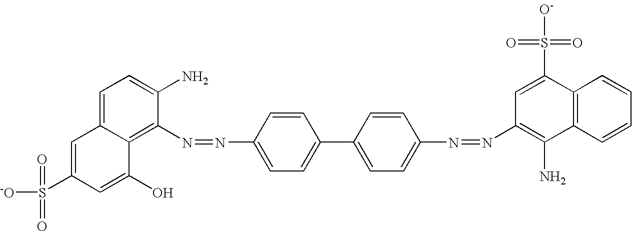 | direct red 13 | 112 | 667 | 4.03 |
| NCI | 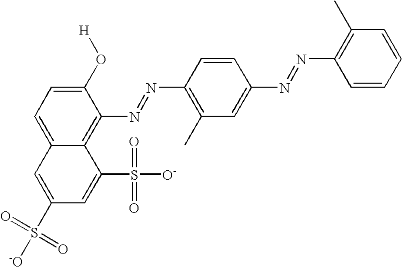 | 45601 | 122 | 589 | 3.93 |
| NCI | 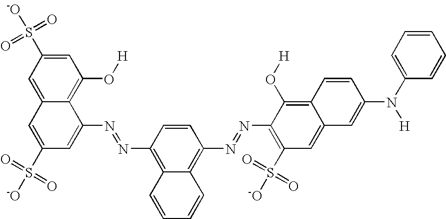 | 75916 | 125 | 797 | 5.23 |
| NCI | 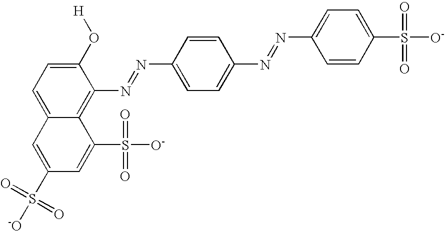 | 65546 | 129 | 590 | 1.78 |
| NCI | 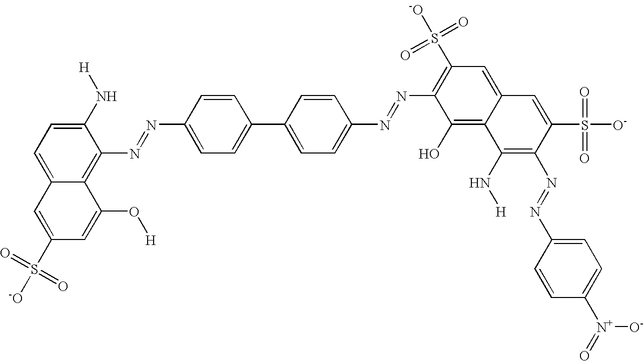 | 65855 | 129 | 911 | 3.86 |
| NCI | 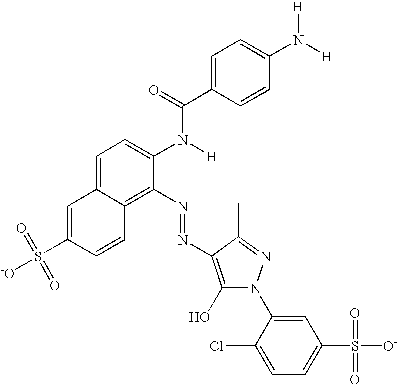 | 75963 | 129 | 655 | 7.25 |
| NCI | 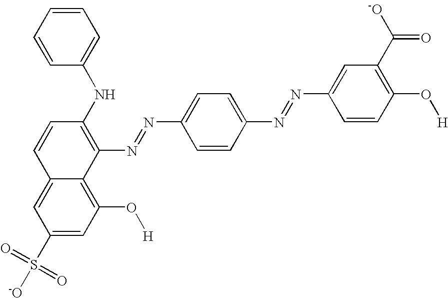 | 45612 | 158 | 582 | 4.99 |
| NCI | 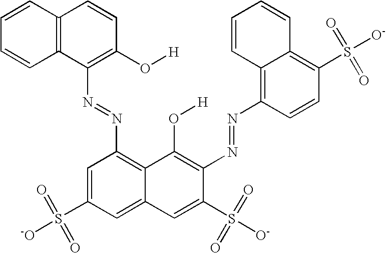 | 8674 | 158 | 706 | 3.63 |
| NCI | 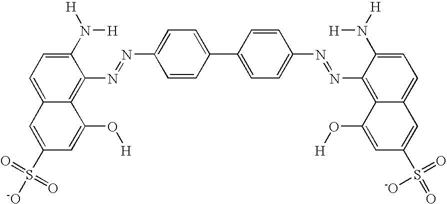 | 75778 | 171 | 683 | 3.64 |
| NCI | 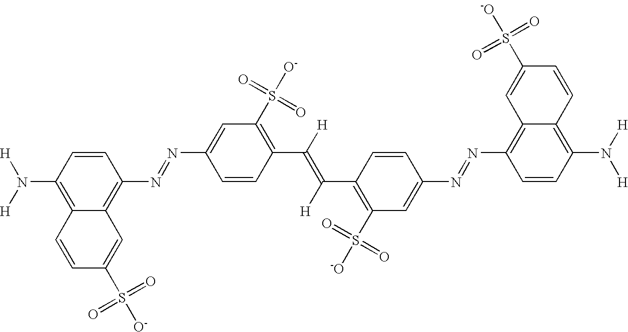 | 34933 | 172 | 835 | 2.51 |
| NIC | 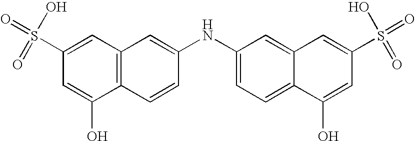 | 1698 | 172 | 462 | 3.03 |
| NCI | 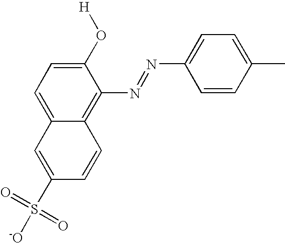 | 7814 | 175 | 341 | 3.27 |
| NCI | 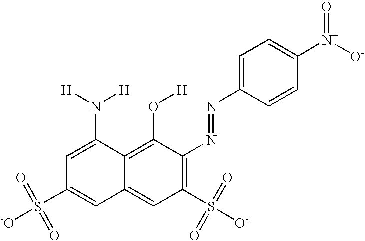 | 45550 | 183 | 466 | 0.67 |
| NCI | 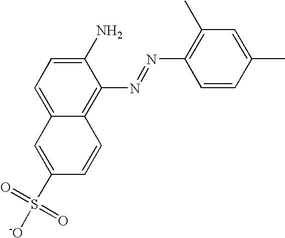 | 45569 | 194 | 354 | 3.26 |
| NCI | 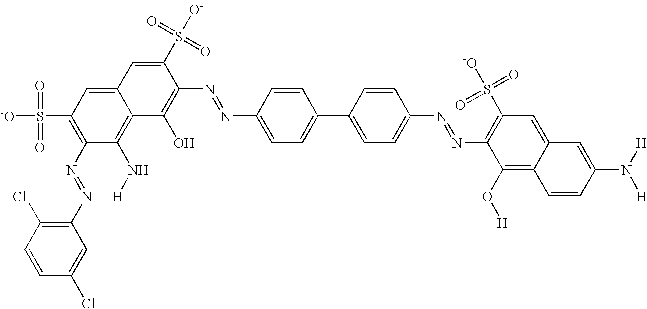 | 77521 | 195 | 935 | 5.13 |
| Sig- ma- Ald- rich | 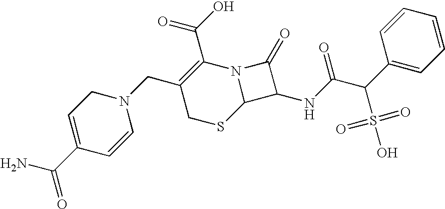 | cefsulodin | 201 | 535 | −1.75 |
| NCI | 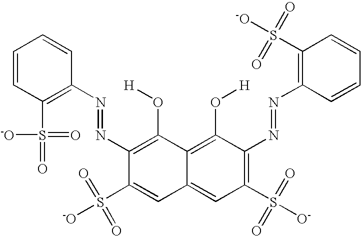 | 174066 | 234 | 685 | 0.07 |
| NCI | 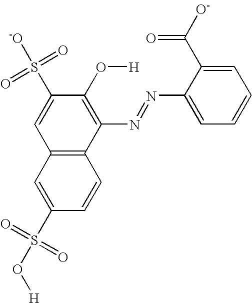 | 12455 | 239 | 450 | 0.88 |
| NCI | 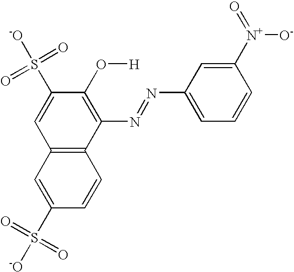 | 45541 | 244 | 451 | 1.49 |
| NCI | 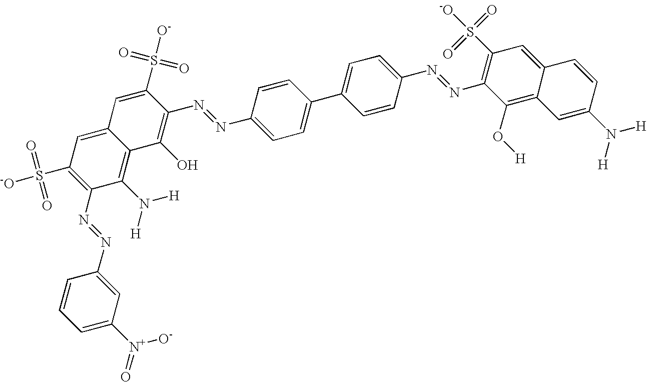 | 79744 | 244 | 911 | 3.86 |
| NCI | 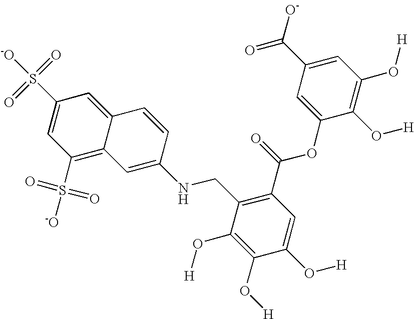 | 42067 | 245 | 635 | −6.06 |
| NCI | 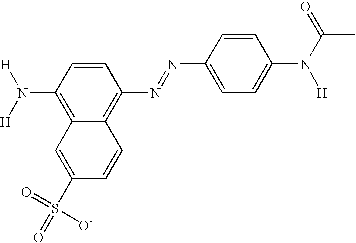 | 45571 | 300 | 383 | 1.84 |
| NCI | 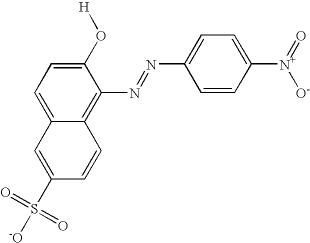 | 45538 | 305 | 372 | 2.81 |
| NCI | 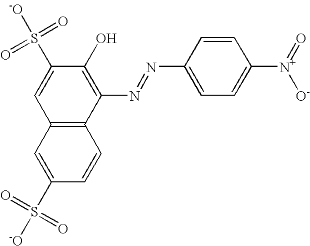 | 45540 | 307 | 451 | 1.49 |
| NCI | 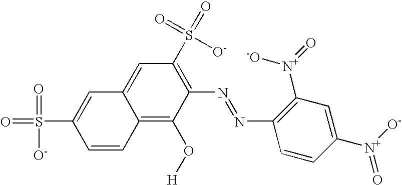 | 9360 | 307 | 496 | 1.45 |
| NCI | 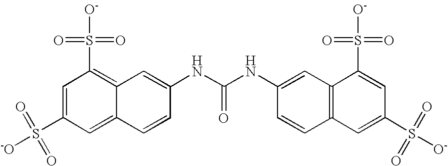 | 12857 | 336 | 629 | −0.52 |
| NCI | 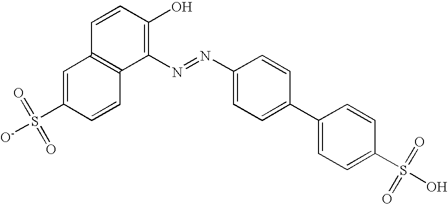 | D726712 | ˜350 | 469 | 5.07 |
| NCI | 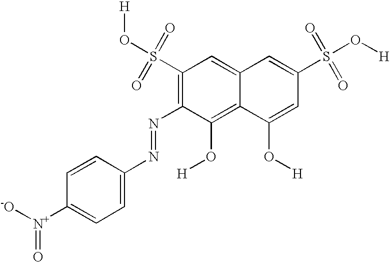 | 45542 | 426 | 469 | 1.10 |
| NCI | 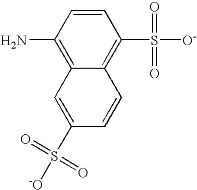 | 7557 | 500 | 301 | −0.47 |
| Rare Ald- rich | 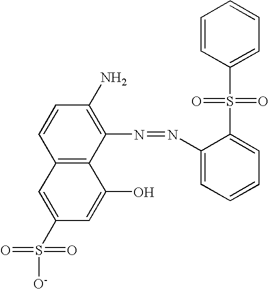 | S321443 | >500 | 483 | 3.64 |
| NCI | 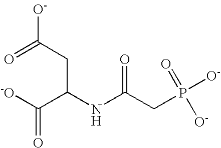 | 224131 | >500 | 251 | −4.97 |
| NCI | 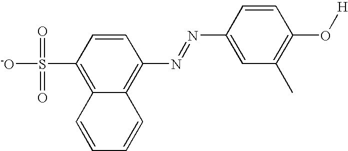 | 45557 | >500 | 341 | 3.27 |
| NCI | 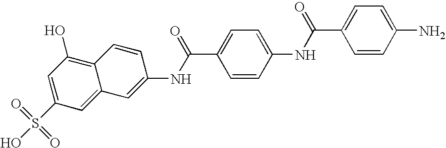 | 1741 | ˜600 | 477 | 2.61 |
| NCI | 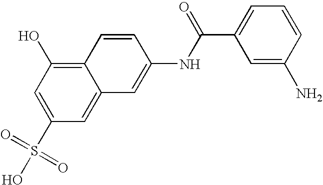 | 1743 | in- active at 75 uM | 358 | 1.80 |
| NCI | 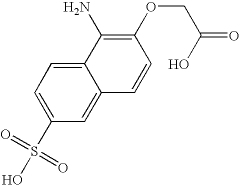 | 16163 | in- active at 75 uM | 297 | 0.53 |
| NCI | 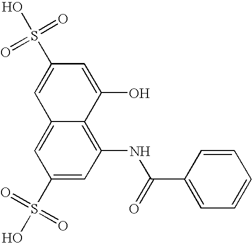 | 16169 | in- active at 75 uM | 423 | 1.76 |
| NCI | 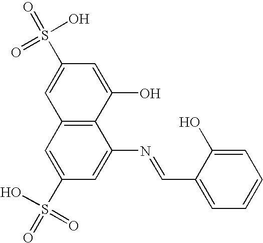 | 88947 | in- active at 75 uM | 423 | 2.44 |
| NCI | 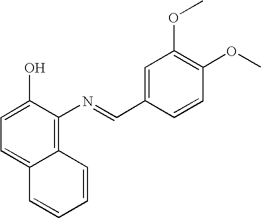 | 17061 | 900 | 307 | 3.46 |
| NCI | 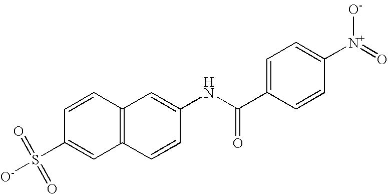 | 37169 | 950 | 371 | 2.33 |
| ACD | 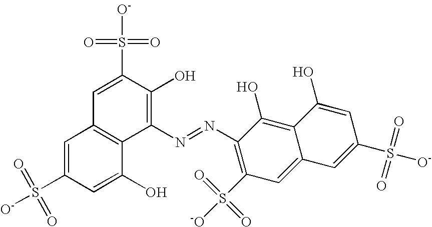 | beryllon II | >1000 | 663 | −1.17 |

TABLE 3
Lead 2 Analogue List

| Li- | | | Kᵢ | |
|---|---|---|---|---|
| brary^{a} | structure | name | (□M) | MW |
| CB | 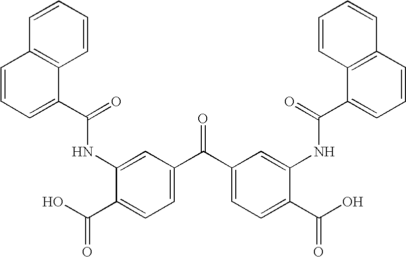 | 181431 compound 2 | 41 | 609 |
| CB | 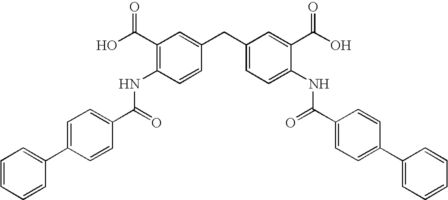 | 473872 compound 2a | 20 | 647 |
| New | 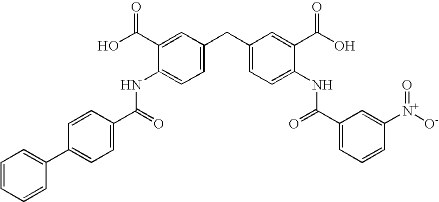 | JLJ-1 | 21 | 615 |
| New | 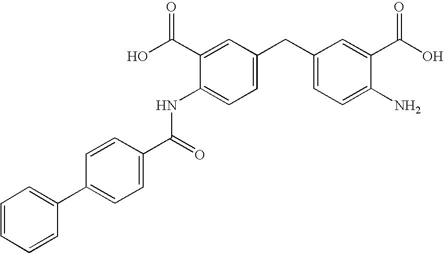 | JLJ-2 | 18 | 466 |
| New | 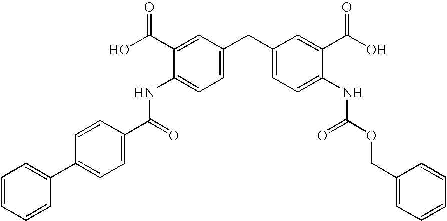 | JLJ-3 | 20 | 600 |
| CB | 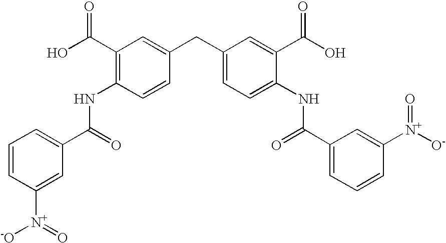 | 467929 compound 2b | 24 | 584 |
| CB | 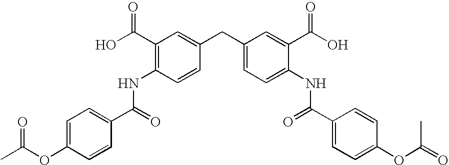 | 534510 | 75 | 611 |
| CB | 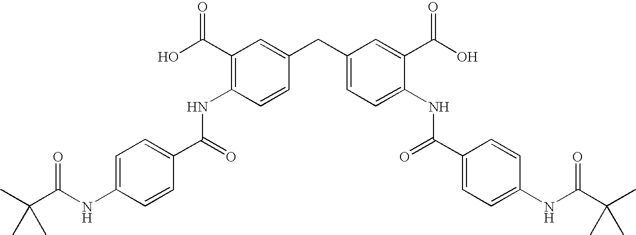 | 540408 | 79 | 695 |
| CB | 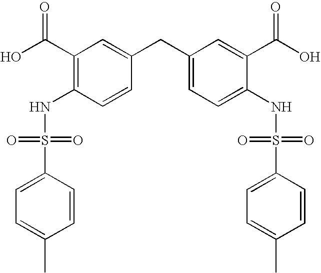 | 180582 | 79 | 595 |
| CB | 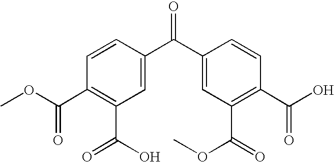 | 180553 | 79 | 386 |
| CB | 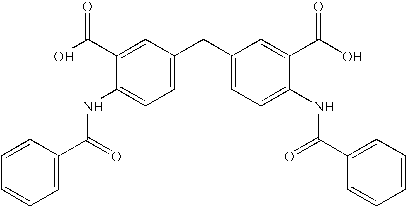 | 186847 | 84 | 494 |
| CB | 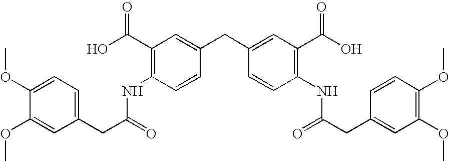 | 477474 | 120 | 643 |
| CB | 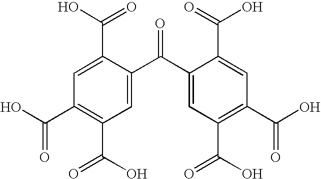 | 152591 | 150 | 446 |
| NCI | 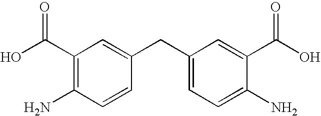 | 37136 | >500 | 286 |
| NCI | 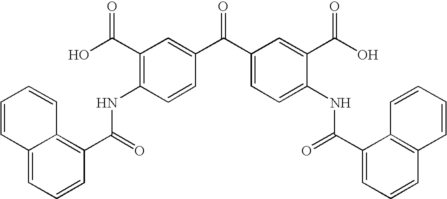 | 202516 | >500 | 609 |
| CB | 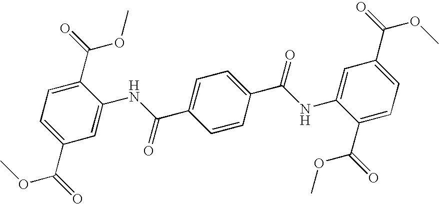 | 039263 | >500 | 549 |
| CB | 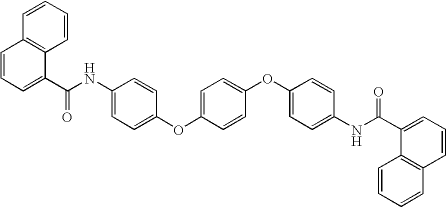 | 181145 | >500 | 601 |
| CB | 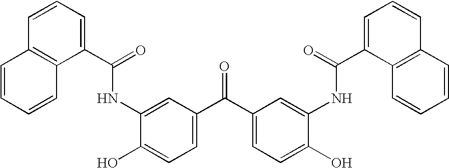 | 181429 | >500 | 553 |
| CB | 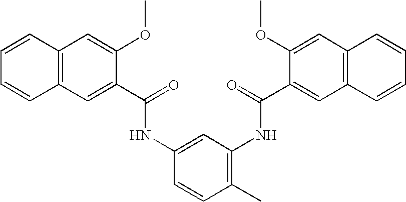 | 205125 | >500 | 491 |
| CB | 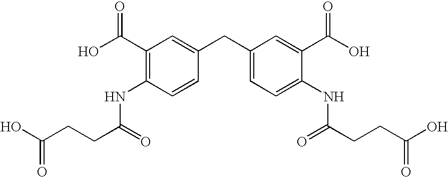 | 224197 | >500 | 486 |

^{a}CB = ChemBridge Corporation;
NCI = National Cancer Institute;
new = synthesized in Shapiro lab (2003)

Some of these analogs have Kᵢ values at least as good as those of the Lead 1 (81 μM) and Lead 2 (41 μM) parent compounds. One of the best inhibitors, a Lead 1 analog also known as Benzopurpurin B, has a Kᵢ for ANG RNase activity of 5 μM. Further, Benzopurpurin B has a higher molecular weight and several additional R groups compared to Lead 1, and may bind to the active site of ANG with a better fit than parent compound Lead 1.

Analog CB-473872 contains biphenyl groups instead of the naphthalene groups and lacks a ketone oxygen on the C atom between the carboxylated amidated phenyl groups of the core, and is more than twice as potent (Kᵢ=20 μM) as Lead 2. This compound, hereafter referred to as Lead 2A, will be used for further rational design, and is likely to have better pharmacological properties than Lead 2, because it has no ketone or other reactive group that would be likely to decrease bioavailability.

### Example 5

### Chemical Designs for Modifications of Leads 1 and 2A

Lead 1 is an azo dye whose substituted naphthalene component is predicted to form four hydrogen bonds with ANG (FIG. 2): two hydrogen bonds between the sulfonate and the side chain of Arg 5, one hydrogen bond between the main-chain oxygen of Arg 5 and the amino group, and another with the His8 imidazole with the same amino group. The azido group sits in the catalytic center with His13 and His114 of ANG on either side, but can potentially hydrogen bond only with His114 at neutral pH (His13 donates its proton to the backbone oxygen of Thr44). Only the OH of the other component of the molecule may interact with ANG, via hydrogen bonds with the side chains of Asp41 and Arg121.

Four modifications of the inhibitor structure are envisioned herein to improve binding. (i) Reduction of the azo to hydrazido (or replacement by an amide as an alternative to reduction) introduces a hydrogen bond with His13 and allows other existing interactions to be optimized. These improvements would outweigh the entropic disadvantage of the additional rotatable bond. (ii) An OH attached to the 6 position of the naphthalene ring can hydrogen bond with Gln12. (iii) Attachment of an electron-withdrawing group at the 6 position of the naphthalene ring is likely to promote tautomerization around the azo bond, which might promote the hydrazo form predicted to interact more favorably with ANG; (iv) Replacing the OH on the biphenyl component with a sulfonate can strengthen the interaction with Arg121. In addition, this lead compound (or minor variations of it) is suited for a combinatorial approach because most of its interactions are predicted to involve the naphthalene half, which may be coupled with a wide variety of aryl amines containing different substituents.

Lead 2A (Lead 2 analog, CB-473872; see FIG. 4 and FIG. 55) contains two benzoic acids which are coupled through a single carbon spacer; each of these is further linked to biphenyl groups via an amide spacer. Contacts between ANG amino acid residues and Lead 2A atoms in the docking model (FIG. 3) include: Arg5 forming two hydrogen bonds with the lower carboxylate; Lys40 forming one hydrogen bond with the upper carboxylate and one hydrogen bond with the amide oxygen; stacking with His114 with both central phenyls; aliphatic portions of Gln 117 and Arg121 sidechains having hydrophobic contacts with upper biphenyls; His13 imidazole forming a hydrogen bond with the upper carboxylate.

Without being limited to any particular mechanism, the model of the complex predicts 5 hydrogen bonds with ANG: one carboxylate forms hydrogen bonds with Lys40; the other carboxylate forms two hydrogen bonds with the guanidino group of Arg5; and one of the amide nitrogens interacts with His13. One of the central phenyl groups of Lead 2A may stack with the His114 imidazole, and form numerous aliphatic contacts. One biphenyl group fits into an area around Asn68, and the other fits near the C-terminal segment of Ang. This area is not well conserved between ANG and RNase A, where in RNase A a tighter hydrophilic nucleobase-binding site would not accommodate buried aromatic surfaces as easily. This presents a potentially powerful means of further tailoring ANG specificity in subsequent analogs.

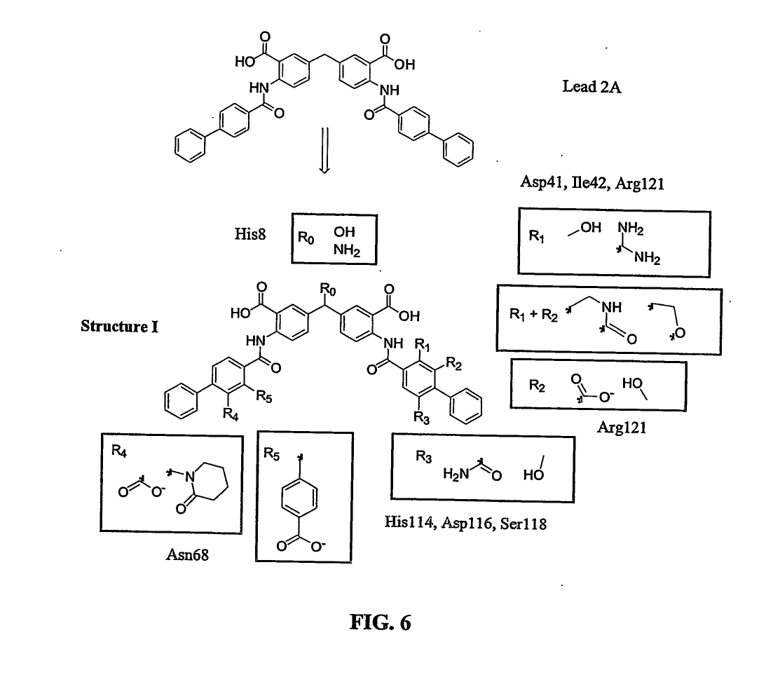

Modifications of Lead 2A (FIGS. 6-8) are designed to improve specificity and affinity for ANG. Without being limited by any particular mechanisms of interactions between derivatives and ANG, the following derivatives are designed based on structural considerations. (i) A hydroxy or an amine group can be substituted at R₀, to form better interactions with His8. (ii) A hydroxymethyl or a methyldiamino group at R₁ may provide superior hydrogen bonding with the ANG amino acid residues Asp41, Ile42 and Arg121. (iii) A hydroxymethyl or a carboxyl group at R₂ may provide superior interaction with Arg 121. (iv) Derivatizing at both R₁ and R₂ either with a formamido-N-ethyl group, to form a 3,4-dihydro-2H-isoquinolin-1-one, or with an ethoxy group, to form a 2,3-dihydrobenzofuran, respectively, may improve interactions with ANG residues Asp41, Ile42 and Arg121. (v) A carboxyamido or hydroxyl substituted at R₃ of Structure I in FIG. 6 may form hydrogen bonds with His 114, Asp116, and Ser118, or may “fill in” the small, hydrophobic gap between the ligand and the solvent-exposed side of the His114 imidazole, providing firm alignment within the extended cavity. (vi) A carboxyl or 2-oxo-N-piperidinyl group can be added at R₄. (vii) a p-benzoyl group added at R₅ may hydrogen bond with the side-chain N of Asn68. Here, a combinatorial approach can be used to help identify the optimal substitutions of groups in place of the upper biphenyl near the obstructed pyrimidine-binding pocket and C-terminal segment of ANG, and to optimize interactions of the lower biphenyl in the purine-binding region.

Derivatives of CB 473872 include a derivative having Structure I (see FIG. 6), and having at least one modification, the modification selected from the group consisting of: R₀ is —H, —NH₂, or —OH; R₁ is a —H, —CH₂OH, or —CH(NH₂)₂; R₂ is —H, —COOH, or —CH₂OH; R₃ is —H, —CH₂OH, or CONH₂; R₄ is —H, —COOH, or 2-oxo-N-piperidinyl; and R₅ is —H or p-benzoyl.

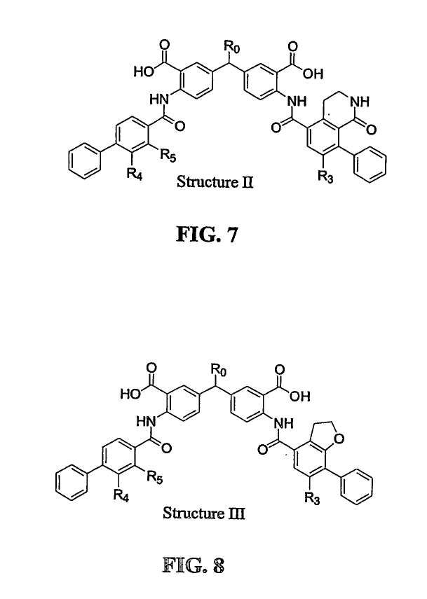

Additional derivatives of CB 473872 include a derivative having Structure II (see FIG. 7) or structure III (see FIG. 8), and having at least one modification, the modification selected from the group consisting of: R₀ is —H, —NH₂, or —OH; R₃ is —H, —CH₂OH, or CONH₂; R₄ is —H, —COOH, or 2-oxo-N-piperidinyl; and R₅ is —H or p-benzoyl group.

These designed modifications are based primarily on predicted increases in affinity of the resulting derivatives of CB-473872 for ANG. However, other features of these molecules (e.g., ADMET properties: absorption; distribution; metabolism; excretion; and toxicology) must also be optimized as part of the process of converting leads into drugs. None of the proposed modifications introduces any chemically reactive groups. Additional changes can be envisioned (e.g. replacements of sulfonates by sulfonamides) that may improve cellular uptake. Moreover, synthesizing compounds having substitutions of nitrogens or oxygens for some of the carbon atoms in the aromatic rings of the lead compounds are envisioned to improve aqueous solubility. With respect to absorption and distribution, that it is possible that ANG inhibitors will not need to enter target cells in order to have therapeutic effects. ANG is a secreted protein and is largely confined to the extracellular space. It may well be sufficient for inhibitors to bind ANG outside the cell.

### Example 6

### Specificity of ANG Inhibitors: Minimizing Inhibition of Other Human RNases

To minimize side effects of potential anti-tumor compounds which are modifications of Lead 1, Lead 2, or Lead 2A (Lead 2 analog, CB-473872), inhibition by these compounds of other general classes of RNase enzymes identified in humans were tested with each inhibitor.

The general classes of RNase are: pancreatic (RNase 1), eosinophil/liver/kidney types (RNases 2, 3, and 6), and RNase 4. It is desirable to minimize cross-reactivity of ANG inhibitors to the enzymatic activities of these other RNases. All small nucleotide-type inhibitors tested in the prior art bind to RNase A and to related human RNases much more tightly than they do to ANG, typically by factors of greater than 100-fold. The docking and modeling studies described herein predicted that the types of small-molecule inhibitors described herein will bind differently to RNase A than to ANG. Results of studies on the effects of mutating various active site amino acids of ANG strongly support the docking predictions. (See Jenkins and Shapiro, 2003.)

Leads 1 (NCI-65828) and 2A (CB-473872), benzopurpurin B, NCI-65820, and CB-467929 were tested as inhibitors of bovine pancreatic RNase A. NCI-65820 and Lead 2A were found to bind ANG 1.5-fold, and 2.3-fold, respectively, to ANG more tightly than RNase A.

These relative affinities indicate the successful capability of design methods to produce a modified drug having more suitable properties, such as increased target specificity, than the parent lead compound.

### Example 7

### Intactness of Total RNA Challenged with Purified RNases in the Presence of Benzopurpurin B (BpB)

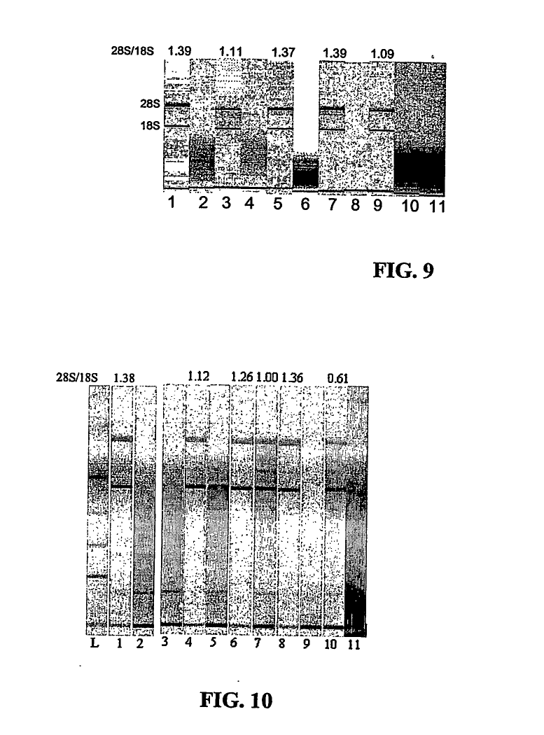

Experiments assessing the inhibition of purified RNases used 8.3 pg/ul bovine RNase A, 1 ng/ul eosinophil-derived neurotoxin (EDN), 1 ng/ul human pancreatic RNase (HPR), 7 ng/ul E. coli RNase I, or 100 pg/ul RNase T1. RNases were pre-incubated with 100 uM Benzopurpurin B (BpB) for 30 sec and then added to 100 ng/ul mouse liver total RNA (Ambion, Inc.) and incubated at 23° C. for an additional 4 min. Samples were quenched in a guanidinium lysis buffer, and the RNA purified via MEGAclear™ MAG-96 and eluted with 20 ul nuclease-free water. The sample (1 μl) was analyzed by 2100 BioAnalyzer software (Agilent) after separation on an RNA LabChip. “Production Source” denotes Ambion, Inc.'s prepared RNA product that is homogenized and purified by organic extraction. FIG. 9 shows that BpB offers significant protection from RNA degradation when challenged by a number of purified RNases, including E. coli RNase I.

### Example 8

### Intactness of Total RNA Challenged with RNases in the Presence of Chembridge Compound #467929

To determine if compound #467929 inhibits various RNases, the inventors subjected total RNA to a panel of RTases in the presence of this compound. The inhibition of several highly purified RNases was tested, namely bovine RNase A (8.3 pg/ul final), HPR (1 ng/ul final), EDN (1 ng/ul), E. coli RNase I (2.5 ng/ul), and RNase T1 (100 pg/ul). All samples were pre-incubated for 30 sec before being added to 100 ng/ul mouse liver total RNA (Ambion) and incubated at 23 C for an additional 4 min. The RNA was then immediately purified via MEGAclear™ MAG-96 and eluted with 20 ul nuclease-free water. The recovered RNA (1 ul) was analyzed by 2100 BioAnalyzer software after separation on an RNA LabChip. Significantly, compound #467929 protected RNA against all purified RNases, save RNase T1 (FIG. 10).

### Example 9

### Inhibition of RT-Based RNase H Activity by Compound #467929

As described above, Chembridge compound #467929 offered protection from several RNA-specific endonucleolytic activities. Assays designed to measure inhibition of RNA cleavage from an RNA-DNA hybrid revealed that this compound was also a relatively potent inhibitor of reverse transcriptase RNase H activity. A 20-mer DNA oligonucleotide was asymmetrically annealed to a 1500 base synthetic RNA to create an RNA:DNA duplex substrate for RNase H. Reactions were initiated with 10 U of MMLV RT in 1×RT buffer (50 mM TrisCl pH 8.3, 75 mM KCl, 3 mM MgCl₂, 5 mM DTT) containing 500 ng/ul RNA:DNA hybrid. All reactions contained 5% DMSO either with or without the small molecule RNase inhibitor. Samples were incubated for 5 min at 37 C, and quenched with EDTA. Products were resolved on an RNA LabChip. Cleavage products were observed as two equally represented RNA species of 1000 and 500 bases. Product length and yield were determined using the 2100 BioAnalyzer software (Agilent). Table 4 details the level of inhibition of MMLV RT RNase H that is observed as a function of the concentration of compounds 467929 and 227726. From these data it is clear that compound #467929 inhibits RT-based RNase H (IC₅₀˜20 uM).

TABLE 4
Level of inhibition of MMLV RT RNase H that Is Observed
as a Function of the Concentration of Compounds
467929 and 227726

| RNase | Inhibitor | |
|---|---|---|
| Inhibitor | Concentration | % Cleavage |
| None | N/A | 36% |
| 467929 | 8 μM | 42% |
| 467929 | 40 μM | 8% |
| 467929 | 80 μM | Not Detectable |
| 227726* | 100 μM | 45% |
| 227726* | 524 μM | 43% |
| 227726* | 1000 μM | 47% |

*Compound 227726 is an N′-[phenyl(4-pyridinyl) methylene]
benzohydrazide with the following structure:

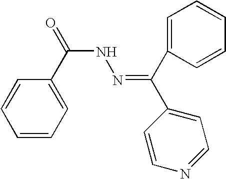

### REFERENCES

The following references, to the extent that they provide exemplary procedural or other details supplementary to those set forth herein, are specifically incorporated herein by reference.
Acharya et al., Proc. Natl. Acad. Sci. USA, 91:2915-2919, 1994.
Chopra et al., Cancer J. Sci. Am., 2:279, 1996.
Chopra et al., J. Cancer Res. Clin. Oncol., 123:167-172, 1997.
Eberle et al., Anticancer Res., 20:1679-1684, 2000.
Etoh et al., Clin. Cancer Res., 6:3545-3551, 2000.
Fett et al., Biochemistry, 24:5480-5486, 1985.
Jenkins and Shapiro, Biochemistry, 42:6674-6687, 2003.
Kao et al., Proc. Natl. Acad. Sci. USA, 99:10066-10071, 2002.
Kelemen et al., Nuc. Acids Res., 27:3696-3701, 1999.
Kumar et al., Biochem. Biophys. Res. Commun., 300:81-86, 2003.
Leonidas et al., J. Mol. Biol., 285:1209-1233, 1999.
Leonidas et al., Protein Sci., 10:1669-1676, 2001.
Morris et al., J. Comp. Chem., 19:1639-1662, 1998.
Olson et al., Cancer Res., 54, 4576-4579, 1994.
Olson et al., Clin. Cancer Res., 7:3598-3605, 2001.
Olson et al., Int. J. Cancer, 98:923-929, 2002.
Olson et al., Proc. Natl. Acad. Sci. USA, 92:442-446, 1995.
Robinson, In: Sustained and Controlled Release Drug Delivery Systems, Marcel Dekker, Inc., NY, 1978.
Russo et al., Meth. Enzymol., 341:629-648, 2001.
Russo et al., Proc. Natl. Acad. Sci. USA, 93:804-808, 1996.
Shapiro et al., Biochemistry, 25:3527-3532, 1986.
Shapiro et al., Biochemistry, 28:1726-1732, 1989.
Shapiro et al., Biochemistry, 28:7401-7408, 1989.
Shapiro et al., Proc. Natl. Acad. Sci. U.S.A, 99(15):10066-71, 2002.
Shapiro et al. Biochemistry, 42:6674-6687, 2003.
Shapiro et al., Proteins, 50(1):81-93, 2003.
Shimoyama et al., Cancer Res., 56:2703-2706, 1996.
Shimoyama et al., Clin. Cancer Res., 5:1125-1130, 1999.
Shimoyama et al., Pancreas, 18:225-230, 1999.
Strydom et al., Biochemistry, 24:5486-5494, 1985.

1-30. (canceled)

31. A method comprising:
(a) obtaining a ribonuclease inhibitor and a composition; and
(b) admixing the ribonuclease inhibitor and the composition to form an admixture, wherein a ribonuclease that may be present in the admixture is inhibited.

32. The method of claim 31, wherein the composition comprises at least one ribonuclease.

33. The method of claim 32, wherein the composition comprises at least two ribonucleases.

34. The method of claim 31, wherein the composition comprises a ribonucleic acid.

35. The method of claim 31, wherein the ribonuclease is an exoribonuclease or an endoribonuclease.

36. The method of claim 31, wherein the ribonuclease is a prokaryotic or eukaryotic ribonuclease.

37. The method of claim 31, wherein the ribonuclease is a mammalian or bacterial ribonuclease.

38. The method of claim 37, wherein the ribonuclease is a mammalian ribonuclease.

39. The method of claim 38, wherein the human ribonuclease is mammalian RNase 1 or human RNase A.

40. The method of claim 37, wherein the ribonuclease the ribonuclease is a bacterial ribonuclease.

41. The method of claim 40, wherein the bacterial ribonuclease is E. coli. RNase 1.

42. The method of claim 31, wherein the ribonuclease inhibitor comprises a structure selected from the group consisting of NCI-65828, NCI 65845, benzopurpurin B, NCI-65841, NCI 79596, NCI-9617, NCI-16224, suramin, direct red 1, NCI-7815, NCI-45618, NCI-47740, prBZBP, NCI-65568, NCI-79741, NCI-65820, NCI-65553, NCI-58047, NCI-65847, xylidene ponceau 2R, eriochrome black T, amaranth, new coccine, acid red 37, acid violet 7, NCI-45608, NCI-75661, NCI-73416, NCI-724225, orange G, NCI 47755, sunset yellow, NCI-47735, NCI-37176, violamine R, NCI-65844, direct red 13, NCI-45601, NCI 75916, NCI-65546, NCI-65855, NCI-75963, NCI-45612, NCI-8674, NCI-75778, NCI-34933, NCI-1698, NCI-7814, NCI-45550, NCI-77521, cefsulodin, NCI-174066, NCI-12455, NCI-45541, NCI-79744, NCI-42067, NCI-45571, NCI-45538, NCI-45540, NCI-9360, NCI-12857, NCI-D726712, NCI-45542, NCI-7557, S321443, NCI-224131, NCI-45557, NCI-1741, NCI-1743, NCI-16163, NCI-16169, NCI-88947, NCI-227726, NCI-17061, NCI-37169, beryllon II, CB-0181431, CB-473872, JLJ-1, JLJ-2, JLJ-3, CB-467929, CB-534510, CB-540408, CB-180582, CB-180553, CB-186847, CB-477474, CB-152591, NCI-37136, NCI-202516, CB-039263, CB-181145, CB-181429, CB-205125, and CB-224197.

43. The method of claim 31, wherein the ribonucleases inhibitor is a derivative of NCI-65828.

44. The method of claim 43, wherein the derivative of NCI-65828 comprises at least one modification selected from the group consisting of: a reduction of the azo to hydrazido, replacement of the azo by an amide, an attachment of a hydroxyl group to position 6 of the naphthalene ring, an attachment of an electron-withdrawing group to position 6 of the naphthalene ring, replacement of a carbon atom in an aromatic ring with a nitrogen or an oxygen, and a replacement of the hydroxyl group on the biphenyl component with a sulfonate.

45. The method of claim 31, wherein the ribonucleases inhibitor is a derivative of NCI-65828.

46. The method of claim 45, wherein the derivative of NCI-65828 comprises at least one modification selected from the group consisting of: an addition of a hydrogen-bonding group and substitution of a hydroxyl group with an anionic group to the biphenyl component.

47. The method of claim 31, wherein the ribonucleases inhibitor is a derivative of CB-473872.

48. The method of claim 47, wherein the derivative of CB-473872 comprises an addition of at least one of a hydrogen-bonding group selected from the consisting of: a hydroxyl, an amino, a methyldiamino, a hydroxyethyl, an ethyl-N-formamido, a carboxyamido, a carboxy, a 2-oxo-N-piperidinyl, and a p-benzoyl.

49. The method of claim 47, wherein the derivative of CB-473872 comprises Structure I, and wherein:
R₀ is —H, —NH₂, or —OH;
R₁ is a —H, —CH₂OH, or —CH(NH₂)₂;
R₂ is —H, —COOH, or —CH₂OH;
R₃ is —H, —CH₂OH, or CONH₂;
R₄ is —H, —COOH, or 2-oxo-N-piperidinyl; and
R₅ is —H or p-benzoyl.

50. The method of claim 47, wherein the derivative of CB-473872 comprises Structure II or Structure III, and wherein:
R₀ is —H, —NH₂, or —OH;
R₃ is —H, —CH₂OH, or CONH₂;
R₄ is —H, —COOH, or 2-oxo-N-piperidinyl;
R₅ is —H or p-benzoyl group.

51. The method of claim 47, wherein the derivative of CB-473872 comprises a replacement of a carbon atom in an aromatic ring with a nitrogen or an oxygen.

52. The method of claim 31, wherein the nuclease inhibitor comprises an aromatic structure.

53. The method of claim 52, wherein the aromatic structure is:

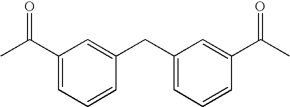

54. The method of claim 31, wherein the nuclease inhibitor comprises a polycyclic aromatic structure.

55. The method of claim 54, wherein the polycyclic aromatic structure is:

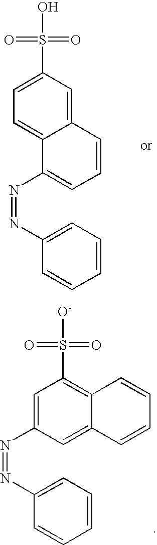

56. The method of claim 31, wherein the nuclease inhibitor comprises the following structure:

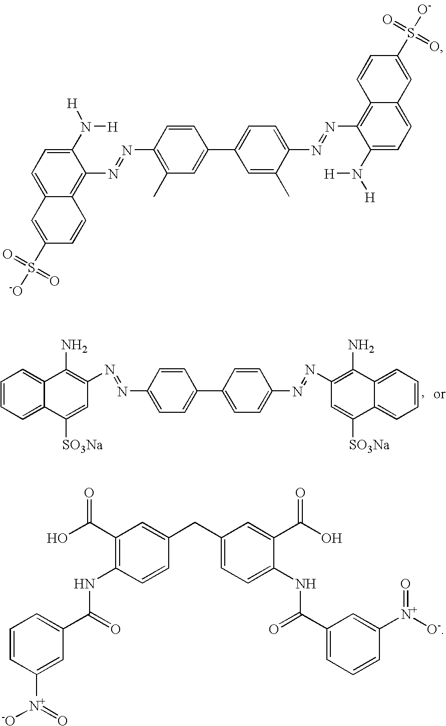

57. A compound comprising a structure selected from the group consisting of NCI-65828, NCI 65845, benzopurpurin B, NCI-65841, NCI 79596, NCI-9617, NCI-16224, suramin, direct red 1, NCI-7815, NCI-45618, NCI-47740, prBZBP, NCI-65568, NCI-79741, NCI-65820, NCI-65553, NCI-58047, NCI-65847, xylidene ponceau 2R, eriochrome black T, amaranth, new coccine, acid red 37, acid violet 7, NCI-45608, NCI-75661, NCI-73416, NCI-724225, orange G, NCI 47755, sunset yellow, NCI-47735, NCI-37176, violamine R, NCI-65844, direct red 13, NCI-45601, NCI 75916, NCI-65546, NCI-65855, NCI-75963, NCI-45612, NCI-8674, NCI-75778, NCI-34933, NCI-1698, NCI-7814, NCI-45550, NCI-77521, cefsulodin, NCI-174066, NCI-12455, NCI-45541, NCI-79744, NCI-42067, NCI-45571, NCI-45538, NCI-45540, NCI-9360, NCI-12857, NCI-D726712, NCI-45542, NCI-7557, S321443, NCI-224131, NCI-45557, NCI-1741, NCI-1743, NCI-227726, NCI-16163, NCI-16169, NCI-88947, NCI-17061, NCI-37169, beryllon II, CB-0181431, CB-473872, JLJ-1, JLJ-2, JLJ-3, CB-467929, CB-534510, CB-540408, CB-180582, CB-180553, CB-186847, CB-477474, CB-152591, NCI-37136, NCI-202516, CB-039263, CB-181145, CB-181429, CB-205125, and CB-224197.
